(19) United States
(12) Patent Application Publication
(10) Pub. No.: US 2017/0368181 A1
(43) Pub. Date: Dec. 28, 2017

(54) Polymeric Nanoparticle

(71) Applicants: The Provost Fellows and Scholars of the College of the Holy and Undivided Trinity of Queen Elizabeth, Dublin (IE); University College Dublin, National University of Ireland, Dublin, Dublin (IE)

(72) Inventors: Owen CORRIGAN, Dublin (IE); Lidia TAJBER, Dublin (IE); Sinead RYAN, Dublin (IE); David BRAYDEN, Dublin (IE); Anita UMERSKA, Dublin (IE)

(21) Appl. No.: 15/599,915

(22) Filed: May 19, 2017

Related U.S. Application Data
(63) Continuation of application No. 13/880,943, filed on Oct. 14, 2013, now abandoned.

(30) Foreign Application Priority Data
Oct. 22, 2010 (GB) 1017889.5

(51) Int. Cl. A61K 47/36; A61K 35/00; A61K 38/17; A61K 9/51; A61K 47/42; A61K 38/23

(52) U.S. Cl. CPC A61K 47/36; A61K 47/42; A61K 38/23; A61K 38/1709; A61K 9/5161; A61K 35/00; A61K 9/5192; A61K 9/5169; A61K 38/1706

(57) ABSTRACT

In a first aspect, the invention provides a polymeric nanoparticle comprising at least one polycationic polymer; at least one polyanionic polymer; and a therapeutically effective amount of at least one therapeutic agent.

In a second aspect, the invention provides a method for the preparation of a polymeric nanoparticle according to the first aspect; the method comprising the steps of: (i)admixing the at least one polyanionic polymer with the at least one therapeutic agent; and (ii) introducing to the mixture of (i), to the at least one polycationic polymer.

In a third aspect, the invention provides a polymeric nanoparticle according to the first aspect of the present invention, or a polymeric nanoparticle prepared according to the second aspect of the present invention; for use in the treatment of an inflammatory and/or arthritic disorder caused by or associated with dysfunctional nuclear receptor signalling.

### FIELD OF THE INVENTION

This invention relates to polymeric nanoparticles comprising at least one polycationic polymer; at least one polyanionic polymer; and a therapeutically effective amount of at least one therapeutic agent. Also disclosed are methods of preparing polymeric nanoparticles, and uses thereof.

### BACKGROUND TO THE INVENTION

Chitosan is a polysaccharide composed of randomly distributed D-glucosamine (deacetylated unit) and N-acetyl-D-glucosamine (acetylated unit), linked by β-(1→4) glycosidic bonds. Chitosan is produced commercially by deacetylation of chitin, a chemically inert polysaccharide found abundantly in nature in the exoskeletons of crustaceans and insects as well as in the cell walls of fungi. Chitosan comprises different length polymer chains. The pKa value of the amino group of chitosan is about 6.5. In acidic media, these amino groups undergo protonation and chitosan therefore becomes polycationic. However, chitosan has low aqueous solubility at neutral pH. The charge density depends on pH and the deacetylation degree. The positive charge density of chitosan influences its reactivity with negatively charged surfaces, such as plasma membranes—thus chitosan is a mucoadhesive polymer. The mucoadhesivity in combination with biocompatibility, non-toxicity, and permeability enhancing properties render chitosan an interesting biomaterial favourable for promoting absorption across mucosal membranes. Chitosan is also degradable in the human body as lysozyme, a-amylase, and chitosanase can degrade chitosan. Any chitosan that is absorbed in the gastro-intestinal tract will likely undergo enzyme degradation to glucosamine and N-acetylglucosamine, which are excreted or used in the amino sugar pool. It has been demonstrated that chitosan has antimicrobial activity and promotes wound healing.

Hyaluronic acid (also called hyaluronan or hyaluronate) is a naturally occurring, mucoadhesive, biocompatible and biodegradable polysaccharide present in the components of extra-cellular matrix of connective tissues and is particularly concentrated in the vitreous fluid of the eye, synovial fluid, umbilical cords and chicken combs. The mucoadhesive character of hyaluronic acid is the result of the establishment of hydrophobic interactions and hydrogen bonds between the hyaluronic acid and the mucus network. Hyaluronic acid is an anionic, nonsulfated glycosaminoglycan composed of repeating disaccharide units containing D-glucuronic acid and D-N-acetylglucosamine linked via alternating β-(1→4) and β-(1→3) glycosidic bonds. In vivo, the polymers of hyaluronic acid can vary in size from 5 to 20000 kDa. Hyaluronic acid is naturally synthesized by hyaluronan synthases, a class of integral membrane proteins. The glucuronic acid and N-acetyloglucosamine units are repeatedly added to the nascent hyaluronic acid molecule by the enzyme as the polysaccharide is extruded via ABC-transporter through the cell membrane into the extracellular space, which results in lengthening of hyaluronic acid molecules.

The inventors have complexed calcitonin in hyaluronic acid and chitosan-based polymeric nanoparticles to yield a complex with enhanced biopharmaceutical and therapeutic properties over the single agents, calcitonin and hyaluronic acid. Additionally, the inventors have prepared polymeric nanoparticles comprising salmon calcitonin (sCT), hyaluronic acid, and protamine, thereby eliminating chitosan, which may have inadvertent competing pro-inflammatory functions, at least in cell-based assays. The new nanoparticles containing calcitonin have potential in treating inflammatory arthritis of joints through down-regulation of the NR4A nuclear receptor family and a range of metalloproteinases following, for example, systemic and intra-articular injection.

### SUMMARY OF THE INVENTION

According to a first aspect of the present invention, there is provided a polymeric nanoparticle comprising at least one polycationic polymer; at least one polyanionic polymer; and a therapeutically effective amount of at least one therapeutic agent.

Optionally, the at least one polycationic polymer, and the at least one polyanionic polymer, are each a biodegradable and biocompatible polymer.

Optionally or additionally, the at least one polycationic polymer, and the at least one polyanionic polymer, are each a polysaccharide. Further optionally, the at least one polycationic polymer, and the at least one polyanionic polymer, are each a mucoadhesive polysaccharide. The at least one polycationic polymer can be a polymer of amino acids, optionally a polymer of amino acids comprising at least one cationic group. Optionally, the at least one polycationic polymer is a poly(amino acid) molecule such as a protein, optionally a poly(amino acid) molecule such as a protein comprising at least one cationic group. Alternatively, the at least one polycationic polymer is a protein, for example, a protamine.

By “polysaccharide” is meant polymers comprising at least two carbohydrate units (either mono- or di-saccharide units) and at least one glycosidic bond extending between the at least two carbohydrate units. The polysaccharide may be linear or branched. Optionally, the polysaccharide is linear.

Optionally, the at least one polycationic polymer, is a weak polycationic polymer. Optionally or additionally, the at least one polyanionic polymer is a weak polyanionic polymer.

Optionally, the at least one polycationic polymer is selected from chitosan and protamine.

Optionally, the at least one polycationic polymer is selected from chitosan. Further optionally, the at least one polycationic polymer is selected from a salt, ester or derivative of chitosan. Optionally, the salt of chitosan is selected from halide salts, for example chloride salts, of chitosan, and glutamate salts of chitosan. Suitable derivatives of chitosan might include carboxymethylated chitosan, an amphiphilic molecule, and N-trimethylated chitosan that is, optionally, soluble at pH 7.

Alternatively, the at least one polycationic polymer is selected from protamine. Further optionally, the at least one polycationic polymer is selected from a salt, ester, amide or other derivative of protamine. Optionally, the salt of protamine is protamine sulphate.

Optionally, the at least one polycationic polymer is chitosan chloride salt. Further optionally, the at least one polycationic polymer is chitosan chloride salt, wherein about 75-90% of the acetyl groups are deacetylated.

Optionally or additionally, the at least one polycationic polymer is chitosan chloride salt and has a molecular weight of about 50-400 kDa. Optionally, the at least one polycationic polymer has a molecular weight of about 50-150 kDa. Alternatively, the at least one polycationic polymer has a molecular weight of about 150-400 kDa.

Optionally, the at least one polycationic polymer is chitosan glutamate salt. Further optionally, the at least one polycationic polymer is chitosan glutamate salt, wherein about 75-90% of the acetyl groups are deacetylated.

Optionally or additionally, the at least one polycationic polymer is chitosan glutamate salt and has a molecular weight of about 50-600 kDa, optionally, 50-400 kDa. Optionally, the at least one polycationic polymer has a molecular weight of about 50-150 kDa. Alternatively, the at least one polycationic polymer has a molecular weight of about 150-600 kDa, optionally, 50-400 kDa.

Optionally, the at least one polyanionic polymer is selected from hyaluronic acid. Further optionally, the at least one polyanionic polymer is selected from a salt of hyaluronic acid. Optionally, the salt of hyaluronic acid is selected from sodium hyaluronic acid and potassium hyaluronic acid.

Optionally, the at least one polyanionic polymer is sodium hyaluronic acid.

Optionally, the at least one polyanionic polymer has a molecular weight of about 5-20,000 kDa. The molecular weight of the sodium hyaluronic acid used in the present invention, before and after sonication may, optionally, be 2300 kDa to 870 kDa before sonication and, after sonication, 290 kDa to 220 kDa.

Optionally, the polymeric nanoparticle has a diameter of about 50-400 nm, optionally 150-300 nm, further optionally 190-250 nm.

Optionally, the at least one polycationic polymer comprises about 7.5-55%, optionally 15-53%, w/w of the polymeric nanoparticle. When the polycationic polymer comprises a derivative of chitosan, optionally, the at least one chitosan comprises about 9-29%, optionally 15-20%, w/w of the polymeric nanoparticle.

Optionally, the polymeric nanoparticle comprises the at least one polyanionic polymer and the at least one polycationic polymer in a ratio of about 2.5:1-10:1 (by weight). Without being bound by theory, it is thought that lower chitosan contents are effective from the therapeutic point of view and that higher chitosan contents may be associated with prolongation of release.

Optionally, the at least one therapeutic agent is water soluble and is optionally selected from low molecular weight chemotherapeutic agents; biological agents; and macromolecular chemotherapeutic agents. An at least one therapeutic agent would be considered water soluble if the agent had a water solubility of more than 0.1 mg/ml at 20° C., optionally more than 0.5 mg/ml at 20° C., further optionally, more than 0.75 mg/ml at 20° C. For example, salmon calcitonin (sCT) has a water solubility of about 1 mg/ml at 20° C.

Optionally, the at least one therapeutic agent is a low molecular weight chemotherapeutic agent selected from nonsteroidal anti-inflammatory agents. Further optionally, the at least one therapeutic agent is a non-steroidal anti-inflammatory agent selected from indomethacin (indometacin), ketoprofen ((RS)2-(3-benzoylphenyl)-propionic acid), aspirin (acetylsalicylic acid), ibuprofen (iso-butyl-propanoic-phenolic acid), and naproxen (naproxen sodium). Alternatively, the at least one therapeutic agent may be selected from steroids, such as, but not limited to, glucocorticoids, such as dexamethasone, prednisolone and prednisone. Further alternatively, the at least one therapeutic agent may be selected from small molecule histamine receptor antagonists for type 1 and type 2 receptors, such as, but not limited to, mepyramine and ranitidine, respectively.

Optionally, the at least one therapeutic agent is a biological agent selected from polynucleotide molecules, such as RNAs and DNAs, and polypeptide molecules.

Optionally, the at least one therapeutic agent is a polynucleotide molecule selected from deoxyribonucleic acid molecules and ribonucleic acid molecules.

Optionally, the at least one therapeutic agent is a polypeptide molecule. Further optionally, the at least one therapeutic agent is a polypeptide hormone molecule, or a propeptide thereof.

By “propeptide” is meant a polypeptide molecule having no or reduced biological function, which, following posttranslational modification, becomes a polypeptide having biological function; and is intended to be synonymous with the terms “protein precursor”, “proprotein”, “preprotein”, or “prepeptide”.

Optionally, the at least one therapeutic agent is a polypeptide hormone molecule selected from the calcitonin family. Alternatively, the at least one therapeutic agent is a propeptide hormone molecule selected from procalcitonin.

Optionally, the calcitonin is selected from human calcitonin; and fish, eel, and bear calcitonin. Further optionally, the calcitonin (CT) is salmon CT (sCT).

According to a second aspect of the present invention, there is provided a method for the preparation of a polymeric nanoparticle according to the first aspect of the present invention; the method comprising the steps of:
(i) admixing the at least one polyanionic polymer with the at least one therapeutic agent; and
(ii) introducing the at least one polycationic polymer to the mixture of (i).

Optionally, the at least one polyanionic polymer is provided in a solution of about 0.1-0.2% (w/v). Further optionally, the at least one polyanionic polymer is provided in an aqueous solution of about 0.1-0.2% (w/v). Without being bound by theory, it is thought that the lower concentrations are not useful in terms of production yield of nanoparticles and that higher concentrations cause formation of micron-sized particles.

Optionally, the at least one polyanionic polymer, or solution thereof, is subjected to agitation prior to the admixing step. Further optionally, the at least one polyanionic polymer, or solution thereof, is subjected to an energy source prior to the admixing step. Still further optionally, the at least one polyanionic polymer, or solution thereof, is subjected to sonication, optionally ultrasound sonication, prior to the admixing step.

Optionally, the at least one polyanionic polymer, or solution thereof, is subjected to ultrasound sonication for at least 10 mins, optionally about 30 mins, further optionally about 30-480 mins, still further optionally about 6 hours.

Optionally, the at least one polycationic polymer is provided in a solution of about 0.1-0.2% (w/v). Further optionally, the at least one polycationic polymer is provided in an aqueous solution of about 0.1-0.2% (w/v). Without being bound by theory, it is thought that the lower concentrations are not useful in terms of production yield of nanoparticles and that higher concentrations cause formation of micron-sized particles.

Optionally, the other of the at least one polycationic polymer and the at least one therapeutic agent in step (ii) is introduced dropwise to the mixture of (i). Optionally or additionally, the introducing step is conducted at room temperature. Further optionally or additionally, the introducing step is conducted under agitation, optionally stirring, further optionally continuous stirring.

Optionally, the method comprises the further step of agitating, optionally stirring, further optionally continuous stirring, the mixture of (ii). Further optionally, the method comprises the further step of agitating, optionally stirring, further optionally continuous stirring, the mixture of (ii) for about 10 minutes.

The method may further comprise the further step of separating aggregates of the polymeric nanoparticle. Optionally, the separating step comprises allowing the mixture of (ii) to stand, optionally not agitated, for about 12 hours.

Optionally, the method further comprises the further step of isolating the polymeric nanoparticle. Optionally, the isolating step comprises centrifugation of the mixture of (ii), or the supernatant obtained from the optional separating step.

Optionally, the at least one polyanionic polymer is provided in an amount of about 0.1-3.0, optionally about 0.5-5.0, mg/ml.

Optionally, the at least one polycationic polymer is provided in an amount of about 0.1-3.0, optionally about 0.5-5.0, mg/ml.

Optionally, the polymeric nanoparticle is prepared under conditions, which are substantially free of surfactant.

According to a third aspect of the present invention there is provided a polymeric nanoparticle according to the first aspect of the present invention, or a polymeric nanoparticle prepared according to the second aspect of the present invention; for use in the treatment of an inflammatory and/or arthritic disorder caused by or associated with dysfunctional nuclear receptor signalling.

Optionally, the nuclear receptor is an orphan nuclear receptor.

Optionally, the nuclear receptor is selected from NR4A1 (nuclear receptor subfamily 4, group A, member 1); NR4A2 (nuclear receptor subfamily 4, group A, member 2); and NR4A3 (nuclear receptor subfamily 4, group A, member 3).

Optionally, the disorder caused by or associated with dysfunctional NR4A1 signalling is a disorder associated with inflammatory responses in macrophages. Such disorders may include inflammatory-arthritis and osteoarthritis. Such disorders may also include inflammatory bowel disease, Crohn's disease, colitis, acute and chronic lung inflammation, and CNS diseases including stroke, acute brain injury, and Alzheimer's disease.

Optionally, the disorder caused by or associated with dysfunctional NR4A2 signalling is a disorder associated with the dopaminergic system of the brain. Further optionally, the disorder caused by or associated with dysfunctional NR4A2 signalling is selected from Parkinson's disease, schizophrenia, manic depression, vascular disease, and cancer.

Alternatively, the disorder caused by or associated with dysfunctional NR4A2 signalling is arthritis, optionally rheumatoid arthritis. Optionally, the disorder caused by or associated with dysfunctional NR4A2 signalling is selected from inflammation-associated arthritis, rheumatoid arthritis, and osteoarthritis.

Optionally, the disorder caused by or associated with dysfunctional NR4A3 signalling is a disorder associated with cell proliferation, differentiation, and metabolism, vascular disease, arthritis, proliferative cancers, and/or atherosclerosis.

Alternatively, there is provided a polymeric nanoparticle according to the first aspect of the present invention, or a polymeric nanoparticle prepared according to the second aspect of the present invention; for use in the treatment of a disorder caused by or associated with dysfunctional matrix metalloproteinase signalling.

Optionally, the matrix metalloproteinase has collagenase activity. Further optionally, the matrix metalloproteinase is matrix metalloproteinase 13. Such disorders include acute lung injury, osteoarthritis, rheumatoid arthritis, and chronic ulcers, optionally requiring matrix re-modelling.

Optionally, the polymeric nanoparticle according to the first aspect of the present invention, or a polymeric nanoparticle prepared according to the second aspect of the present invention; is provided for use in the treatment of arthritis.

Optionally, the arthritis is rheumatoid arthritis. Further optionally, the arthritis is selected from inflammation-associated arthritis, inflammatory arthritis, rheumatoid arthritis, and osteoarthritis.

Optionally, the polymeric nanoparticle according to the first aspect of the present invention, or a polymeric nanoparticle prepared according to the second aspect of the present invention; is provided for use in the treatment of disorders selected from arthritis, chronic ulcers requiring matrix re-modelling, and inflammatory bowel diseases such as Crohn's and/or colitis.

Optionally, the polymeric nanoparticle according to the first aspect of the present invention, or a polymeric nanoparticle prepared according to the second aspect of the present invention; is provided for use in the treatment of disorders selected from postmenopausal osteoporosis, hypercalcaemia, Paget's disease, bone metastases, phantom limb pain, and spinal stenosis.

### BRIEF DESCRIPTION OF THE DRAWINGS

Embodiments of the present invention will be described, with reference to the accompanying drawings, in which:

FIG. 1A is a scanning electron micrograph depicting the morphology of a polymeric nanoparticle comprising at least one polycationic polymer; and at least one polyanionic polymer;

FIG. 1B is a graph plotting the size distribution of a polymeric nanoparticle comprising at least one polycationic polymer; and at least one polyanionic polymer; wherein the average diameter is in the 100-300 nm range;

FIG. 2 is a graph plotting Fourier transform infrared spectroscopic analysis of sodium hyaluronic acid (HA), chitosan CL213 (CS), and polymeric nanoparticles (NP) comprising at least one polycationic polymer and at least one polyanionic polymer;

FIG. 3A is a scanning electron micrograph depicting the morphology of a polymeric nanoparticle comprising sodium hyaluronic acid and chitosan glutamate salt;

FIG. 3B is a graph plotting the size distribution of a polymeric nanoparticle comprising sodium hyaluronic acid and chitosan glutamate salt;

FIG. 4 is a concentration-response curve depicting the effects of polymeric nanoparticles comprising hyaluronic acid and chitosan on collagen-induced (2 μg/ml) platelet aggregation;

FIG. 5 is a graph comparing the in vitro bioactivity of sCT containing polymeric nanoparticles to native sCT for stimulation of cAMP secretion on T47D breast cancer cells over 60 min; wherein I, II and III are selected complex-released sCT from different batches and IV is a negative control, saline;

FIG. 6A is a graph illustrating human chondrosarcoma cells SW1353 pre-treated with (B) IL-1β and combinations of polymeric nanoparticles (100 nM) or the positive control, dexamethasone (100 nM) for 1.5 hours followed by stimulation with the pro-inflammatory signalling molecule, PGE₂ (1 μM);

FIG. 6B is a graph illustrating human chondrosarcoma cells SW1353 treated with IL-1β and combinations of polymeric nanoparticles (100 nM) or dexamethasone (100 nM) for 1.5 hours; and demonstrating the enhanced effects of the combined sCT with HA particulates compared to either agent alone in their ability to prevent both NR4A2 and MMP 13 mRNA expression as determined by RT-PCR;

FIG. 7 refers to the normalised capacity of low concentration of sCT, HA and the combination thereof to reduce expression of NR4A2 in SW1353 cells pre-treated with the inflammatory stimulator, prostaglandin E2 (PGE2); wherein synergistic effects are seen when the two agents are combined at the same concentrations as those used for either agent alone;

FIGS. 8A-B are electron transmission micrographs of a polymeric nanoparticle comprising hyaluronic acid, chitosan, and sCT (sCT content: 0.09mg/mg of powder);

FIG. 9 is a graph depicting percent sCT released from polymeric nanoparticles comprising hyaluronic acid, chitosan, and sCT;

FIGS. 10A-B are graphs depicting the beneficial effects of a polymeric nanoparticle comprising hyaluronic acid, chitosan, and sCT delivered in a single injection by the intra-articular (IA) route to the knee on reducing knee diameter over time (FIG. 10A) and the clinical score of the ankle joint (FIG. 10B) when administered to the K/BxN mouse model of osteoarthritis; wherein all formulations reduced knee diameter; however comparable reductions in swelling were seen only with the nanoparticle formulation to that of the positive control and gold standard, dexamethasone; and wherein, importantly, the reduction in diameter seen with the nanoparticles was better than for sCT alone, the overall dose of sCT being the same in both groups; and wherein the clinical score was unchanged by any treatment as that refers to all four paws, for which 3 were untreated;

FIGS. 11A-D illustrate histological analysis of knee joints from K/BxN induced arthritis mouse model stained with haemotoxylin and eosin (H&E); wherein the repair of bone architecture can be seen with both the nanocomplexes containing sCT and also with the gold standard, dexamethasone; FIG. 11A illustrates naïve no arthritis induced; FIG. 11B illustrates arthritis induced, bone erosion (BE), certilage erosion (CE) andsynovitis (SV); FIG. 11C illustrates arthritis induced treated with sCT nanocomplex; and FIG. 11D illustrates arthritis induced treated with dex;

FIGS. 12A-C illustrate histological analysis of ankle joint of K/BxN model with Saffarin O staining of the cartilage; and demonstrating the effectiveness of the sCT:HA nanoparticle therapy since there is evidence of cartilage regeneration; and wherein, importantly, the polymeric nanoparticle gave greater cartilage staining than a simple ad-mixture of sCT with HA, thus illustrating the importance of having a nanoparticle composition; FIG. 12A shows a normal healthy joint with no cartilage degradation; FIG. 12B shows cartilage erosion (CE) and synovitis (SV); FIG. 12C shows the cartilage is preserved in a nanocomplex;

FIG. 13 illustrates the influence of sonication time on the molecular weight of HA and viscosity of its 0.1% solution;

FIGS. 14A-B illustrate the influence of HA/CS mixing ratio, total polymer concentration and type of chitosan salt on: mean particle size (FIG. 14A) and zeta potential of HA/CS NPs (FIG. 14B);

FIGS. 15A-B illustrate the influence of molecular weight of HA on: mean particle size (FIG. 15A) and zeta potential of polymeric nanoparticles with different HA/CS mixing ratios (FIG. 15B);

FIG. 16 illustrates pH versus sonication time for nanoparticles containing the following HA/chitosan mass mixing ratios: 5 (denoted “10+2”)—grey (within each set of 3 columns, this is the left hand column), 2.5 (Denoted “10+4”)—black (within each set of 3 columns, this is the middle column), 1 (denoted “5+5”)—white (within each set of 3 columns, this is the right hand column);

FIG. 17 illustrates a TEM micrograph of HA/CL113 nanoparticles (concentration of CL113 and sonicated (t=2 hr) HA solutions was 0.1%, and the HA/CL113 mass ratio was 5;

FIGS. 18A-B are graphs depicting percent sCT released from polymeric nanoparticles comprising hyaluronic acid, chitosan and sCT (formulation codes as in Table 4F);

FIGS. 19A-B are graphs depicting percent sCT released from polymeric nanoparticles comprising hyaluronic acid, protamine and sCT;

FIG. 20 illustrates the expression levels of NR4A1, NR4A2 and NR4A3 in the ankle joints of the K/BxN serum induced BALB/c mice 6 days after serum transfer;

FIG. 21 illustrates the expression levels of NR4A2 in the ankle joints in sera-induced mice with treatment groups of sCT, sCT-HA NP, and dexamethasone administered by intra-peritoneal injection; wherein controls included a naïve (no inflammation) and serum induced group with no treatment; and wherein all treatments (sCT, sCT:HA NP, and dex) were seen to reduce NR4A2 expression; whereby sCT:HA NP was superior to ad-mixed sCT:HA in reducing NR4A2 expression levels and also comparable to the positive control dexamethasone;

FIGS. 22A-E illustrate histological analysis of knee joints from K/BxN induced arthritis mouse model stained with haemotoxylin and eosin (H&E); wherein the repair of bone architecture can be seen with both the nanocomplexes containing sCT and also with the gold standard, dexamethasone;

FIGS. 23A-C illustrate the histology analysis of ankle joint of K/BxN model with Saffarin O staining of the cartilage; wherein serum alone shows severe bone and cartilage erosion and synovitis infiltration; and wherein Naïve shows a normal healthy joint with no cartilage degradation; whereby serum with treatment of sCT:HA in a nanocomplex shows the cartilage is preserved; and

FIG. 24 illustrates the histological score of the knee joint (left knee; no treatment, right knee; treatment) as scored by an observer blinded to the treatment (intra-articular delivery) under 3 different criteria (i) inflammation (synovitis infiltration and pannus formation), (ii) cartilage erosion and (iii) bone erosion on a scale of 0-3 (0, none; 1, minimal invasion of meniscus; 2, partial invasion of meniscus; 3, severe invasion of meniscus).

### EXAMPLES

Embodiments of the present invention will now be described, by way of non-limiting example.

### Example 1

### A Polymeric Nanoparticle Comprising Sodium Hyaluronic Acid; and Chitosan Chloride Salt

The hyaluronic acid and chitosan used in this preparation were sodium hyaluronic acid from Fluka (HA(F)) and chitosan chloride salt (CL213, molecular weight of 150-400 kDa).

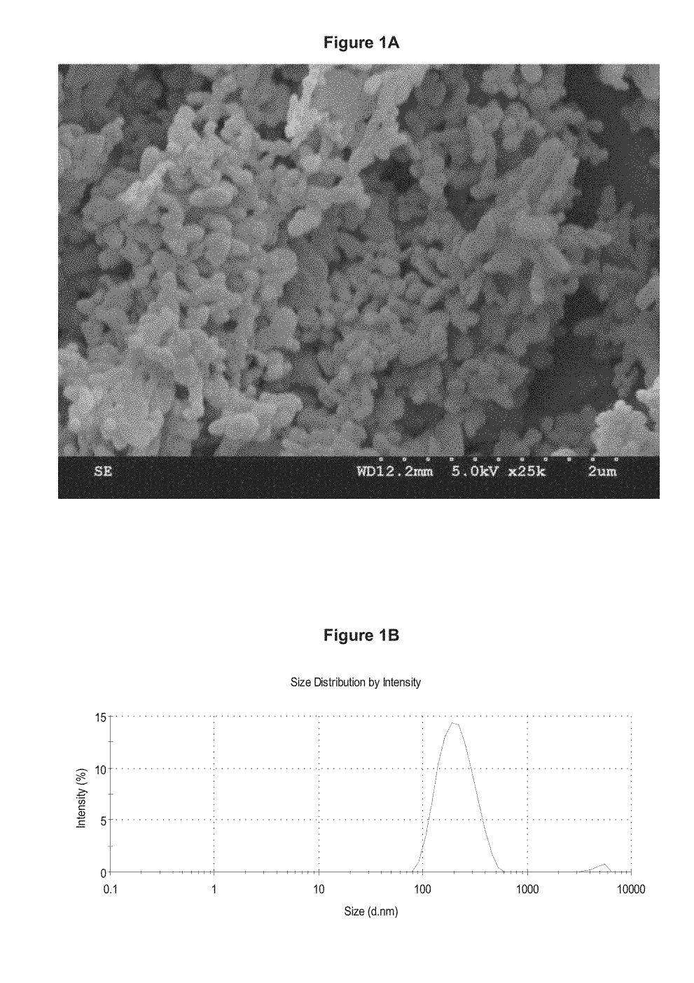

The particle size of the resultant HA(F)/CL213 particles was measured with a Malvern Zetasizer and was 198±11 nm. The zeta potential was 1.64±0.52 mV. The chitosan loading in the HA(F)/CL213 nanoparticles was 42.7±9.7% w/w, determined by ninhydrin analysis. The physical properties of HA(F)/CL213 nanoparticles are listed in Table 1 and the SEM picture with size distribution is shown in FIGS. 1A and 1B, respectively.

TABLE 1
Physical properties of HA/CS nanoparticles

| | z-Average | | Chitosan | Zeta |
|---|---|---|---|---|
| | size (nm) | Size width | content % | potential |
| Name | (n = 3) | (nm) (n = 3) | (w/w) (n = 3) | (mV) (n = 5) |
| HA(F)/CL213 | 198 ± 11 | 99.1 ± 14.3 | 42. 7 ± 9.7 | 1.64 ± 0.52 |
| NP | | | | |

(Samples represent the mean value ± standard deviation)

Referring to FIGS. 1A and 1B, it can be seen that the polymeric nanoparticles are in the nano size range and have relatively irregular shapes.

Fourier transform infrared spectroscopic analysis (FTIR) of the resultant HA/CS nanoparticles was performed and compared to each of sodium hyaluronic acid (HA) and chitosan CL213 (CS) to identify the presence of hyaluronic acid and chitosan in the polymeric nanoparticles (NP).

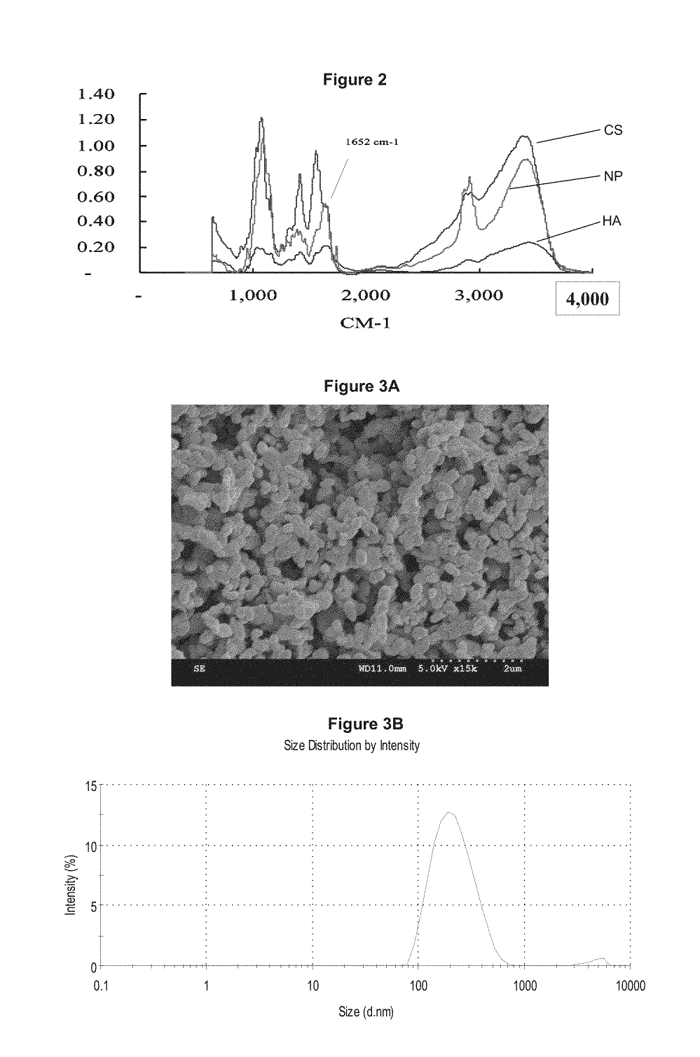

Referring to FIG. 2, it can be seen that sodium hyaluronic acid (HA) has an absorption peak at 1652 cm⁻¹, which is characteristic of the C═O stretch of carboxylic acid in hyaluronic acid polymers. Polymeric nanoparticles also have this absorption peak at 1625 cm⁻¹, indicating the presence of hyaluronic acid in polymeric nanoparticles (NP). The absorption at 2930 cm⁻¹ is characteristic of a stretch of —CH₃groups. The increased intensity of this band in the polymeric nanoparticle system at 2930 cm⁻¹ compared to sodium hyaluronic acid alone (HA) and chitosan CL213 alone (CS) could be attributed to interactions between the polymers and as a result, shifting peaks exposing the band 2930 cm⁻¹. The FTIR result indicated the presence of hyaluronic acid and chitosan in polymeric nanoparticles. Sodium sulphate was used in production of the polymeric nanoparticles in Example 1, and acted as a desolvation reagent to facilitate the phase separation.

### Example 2

### A Polymeric Nanoparticle Comprising Sodium Hyaluronic Acid and Chitosan Chloride Salt; and a Polymeric Nanoparticle Comprising Sodium Hyaluronic Acid and Chitosan Glutamate Salt

Highly purified sodium hyaluronic acid (NovaMatrix) (HA) was used to prepare polymeric nanoparticles with the chitosan salts (CS): CL213, CL113 (chloride salt of chitosan, molecular weight of <150 kDa), G213 (glutamate salt of chitosan, molecular weight of 150-400 kDa), and G113 (glutamate salt of chitosan, molecular weight of <150 kDa). The difference with sodium hyaluronic acid (Fluka) is the low lipopolysaccharide (LPS) level of the NovaMatrix grade, which is lower than 2.5 EU/g (endotoxin units per gram) as claimed by the manufacturer. According to the U.S. Food and Drug Administration guideline, the upper limit of the pyrogen level is 5.0 endotoxin units (EU)/kg (body weight) per injection. The physical properties of the resultant nanoparticles are listed in Table 2.

TABLE 2
Physical properties of polymeric nanoparticles

| | z-Average | | Chitosan | Zeta |
|---|---|---|---|---|
| | Size (nm) | Size width | content % | Potential |
| Name | (n = 3) | (nm) (n = 3) | (w/w) (n = 3) | (mV) (n = 5) |
| HA/CL113 | 171.2 ± 21.3 | 70.2 ± 12.1 | 43.2 ± 5.1 | 1.55 ± 0.44 |
| NP | | | | |
| HA/CL213 | 183.8 ± 31.2 | 85.6 ± 27.3 | 45.8 ± 6.7 | 2.12 ± 0.29 |
| NP | | | | |
| HA/G113 | 169.8 ± 50.7 | 71.6 ± 16.6 | 45.1 ± 3.3 | 1.34 ± 0.72 |
| NP | | | | |
| HA/G213 | 176.5 ± 38.8 | 78.4 ± 12.2 | 44.8 ± 7.9 | 1.28 ± 0.31 |
| NP | | | | |

(Samples represent the mean value ± standard deviation)

The prepared nanoparticles were tested for their potential use as adjuvants in vaccine formulation. Table 3 outlines the content of lipopolysaccharides (LPS) in the nanoparticles produced. EM pictures with size distribution of a polymeric nanoparticle comprising sodium hyaluronic acid and chitosan glutamate salt is shown in FIGS. 3A and 3B, respectively.

TABLE 3
Content of lipopolysaccharides (LPS)
in the polymeric nanoparticles

| | | | | LPS conc | LPS/sample |
|---|---|---|---|---|---|
| Sample | Conc. | | LPS conc. | (EU/ml) | (ng/mg) |
| HA/CL213 | 100 | μg/ml | 3.303679 | 33.03679 | 3.3 |
| | 10 | μg/ml | 0.609751 | 60.97514 | 6.1 |
| | 1 | μg/ml | −0.10435 | not detectable | |
| HA/CL113 | 100 | μg/ml | 5.032866 | 50.32866 | 5.3 |
| | 10 | μg/ml | 0.065214 | 6.52136 | 0.7 |
| | 1 | μg/ml | −0.22664 | not detectable | |
| HA/G213 | 100 | μg/ml | 0.406376 | 4.06376 | 0.4 |
| | 10 | μg/ml | −0.19596 | not detectable | |
| | 1 | μg/ml | −0.26828 | not detectable | |
| HA/G113 | 100 | μg/ml | 1.433103 | 14.33103 | 1.4 |
| | 10 | μg/ml | 0.133086 | 13.30862 | 1.3 |
| | 1 | μg/ml | −0.31929 | not detectable | |
| HA control | 100 | μg/ml | −0.1965 | not detectable | |
| | 10 | μg/ml | −0.29107 | not detectable | |
| | 1 | μg/ml | −0.25963 | not detectable | |
| HA (F)/CL213 | 100 | μg/ml | 1.878411 | 18.78411 | 1.9 |
| | 10 | μg/ml | 0.012145 | 1.21448 | 0.1 |
| | 1 | μg/ml | −0.14735 | not detectable | |

### Example 3

### Platelet Compatibility of Polymeric Nanoparticles Comprising Hyaluronic Acid (HA) and Chitosan (CS)

The hyaluronic acid used in this preparation was sodium hyaluronate from Fluka and chitosan was the chloride salt (CL213, molecular weight of 150-400 kDa).

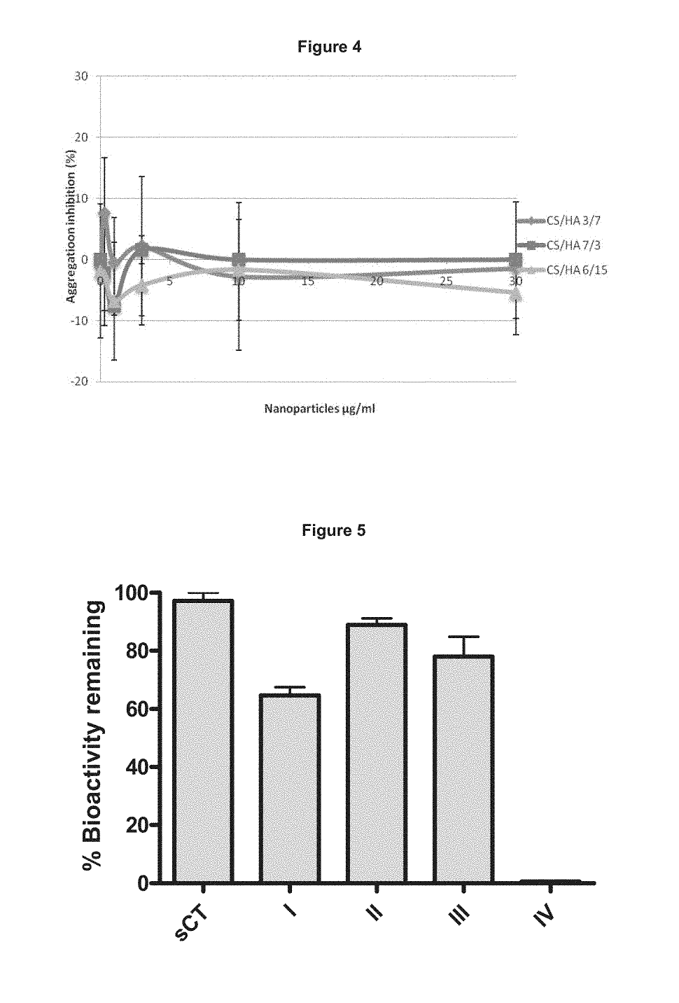

Three batches of the polymeric nanoparticles comprising hyaluronic acid and chitosan at the ratios of 3:7 (CS/HA 3/7), 7:3 (CS/HA 7/3) and 6:15 (CS/HA 6/15) (by weight) were screened to investigate their effects on human platelet aggregation in vitro. None of the polymeric nanoparticles caused platelet aggregation at concentrations 10-30 μg/ml. The effects of polymeric nanoparticles to influence collagen-induced platelet aggregation were also investigated. For these studies platelet aggregation was initiated by the addition of collagen (2 μg/ml). Referring to FIG. 4, it can be seen that the concentrations of polymeric nanoparticles used (10-30 μg/ml) did not prevent aggregation of platelets triggered by collagen.

### Example 4

### A Polymeric Nanoparticle Comprising Hyaluronic Acid; Chitosan; and sCT

Polymeric nanoparticles with different proportions (mass ratios) of hyaluronic acid (HA, Sigma or NovaMatrix, Norway) and e.g. chloride salt of chitosan (CL113, Novamatrix) (CS) were prepared using a very mild technique of polyelectrolyte complex formation. These polymeric nanoparticles were loaded with salmon calcitonin (sCT) as a model therapeutic agent. The sodium hyaluronic acid solution prepared at a concentration of 0.1% w/v in deionised water was ultrasonicated (90 minutes, power 80%), and an appropriate amount of sCT (as defined herein below) was dissolved in that solution prior to the formation of polymeric nanoparticles. CS solution (0.1% w/v in deionised water) was added drop wise to the HA or HA/sCT solutions at room temperature under magnetic stirring. Stirring was maintained for 10 minutes to allow the complete formation of the polymeric nanoparticle system. Polymeric nanoparticles were instantaneously obtained upon the addition of the CS solution to HA or HA/sCT solution, but in some cases micron-sized particles were formed. The encapsulation efficiency of sCT in the nanoparticles was calculated by the difference between the total amount of sCT used to prepare the loaded polymeric nanoparticles and the remaining amount of free sCT in the aqueous medium. The amount of free sCT was determined in the supernatant by HPLC technique following the separation of polymeric nanoparticles from aqueous medium by centrifugation. When CS solution was added to HA solution under stirring conditions (no ultrasounds), in most cases micron-sized particles were formed.

The properties of the nanoparticles (such as particle size, polydispersity index and zeta potential) depend on their composition. As a function of increasing amount of HA and decreasing amount of CL113 the zeta potential of the particles decreases, then undergoes an inversion from positive to negative, and then with a further increase of HA it becomes more negative. This may be due to the fact that at first, an insoluble polyelectrolyte complex between HA and CL113 is formed, and then, with the further increase in the amount of HA, the excess of HA is bound to the particle surface by weaker, reversible interactions, what causes the decrease in the zeta potential.

TABLE 4A
GPC and viscosity measurements confirmed
depolymerisation of HA

| | Molecular weight | Dynamic viscosity |
|---|---|---|
| | [kDa] | [mPa · s] |
| Non-sonicated HA | 1590 ± 720 | 14.63 ± 0.09 |
| Ultrasonicated HA (uHA) | 255 ± 31 | 2.08 ± 0.05 |

No significant differences in FTIR were observed between the two types of HA indicating no chemical changes in the polymer structure.

TABLE 4B
Properties of polymeric nanoparticles comprising HA and CS.

| | | | Mean | | Zeta |
|---|---|---|---|---|---|
| HA | CL113 (CS) | | particle | Polydispersity | potential |
| [mg/ml] | [mg/ml] | pH | size [nm] | index | [mV] |
| 0.952 | 0.048 | 7.03 | 480 ± 179 | 0.251 ± 0.064 | −49.7 ± 6.3 |
| 0.909 | 0.091 | 6.88 | 393 ± 150 | 0.236 ± 0.063 | −47.3 ± 6.3 |
| 0.833 | 0.167 | 6.80 | 310 ± 116 | 0.204 ± 0.077 | −41.4 ± 3.9 |
| 0.714 | 0.284 | 6.69 | 202 ± 45 | 0.158 ± 0.058 | −33.5 ± 2.8 |
| 0.625 | 0.375 | 6.62 | 176 ± 23 | 0.106 ± 0.037 | −25.6 ± 3.2 |
| 0.556 | 0.444 | 5.85 | 274 ± 69 | 0.150 ± 0.063 | +29.1 ± 9.2 |
| 0.500 | 0.500 | 5.33 | 303 ± 65 | 0.137 ± 0.043 | +39.9 ± 3.3 |
| 0.286 | 0.714 | 4.93 | 892 ± 142 | 0.304 ± 0.072 | +53.1 ± 2.3 |

Table 4B shows that, with the increase in the HA/CS ratio, the size and polydispersity index of polymeric nanoparticles are at first decreasing (the size decreases from 900 nm to 180 nm) and then, after inversion of zeta potential, they are increasing again.

TABLE 4C
Properties of polymeric nanoparticles
comprising HA, CS, and sCT (100 μg/ml)

| | | | | Encapsulated | |
|---|---|---|---|---|---|
| sCT | HA | CL113 | | quantity | % of |
| [mg/ml] | [mg/ml] | [mg/ml] | pH | [μg/ml] | encapsulation |
| 0.100 | 0.714 | 0.284 | 6.61 | 8.47 ± 4.04 | 8.42 ± 4.03 |
| 0.100 | 0.833 | 0.167 | 6.64 | 7.33 ± 7.61 | 7.25 ± 7.56 |
| 0.100 | 0.909 | 0.091 | 6.67 | 4.50 ± 4.50 | 4.43 ± 4.45 |

| | | | Mean | | Zeta |
|---|---|---|---|---|---|
| sCT | HA | CL113 | particle | Polydispersity | potential |
| [mg/ml] | [mg/ml] | [mg/ml] | size [nm] | index | [mV] |
| 0.100 | 0.714 | 0.284 | 206 ± 18 | 0.139 ± 0.016 | −33.2 ± 2.9 |
| 0.100 | 0.833 | 0.167 | 294 ± 50 | 0.198 ± 0.036 | −40.3 ± 3.4 |
| 0.100 | 0.909 | 0.091 | 398 ± 91 | 0.231 ± 0.049 | −44.6 ± 5.0 |

Table 4C shows that HA/CS polymeric nanoparticles loaded with sCT (100 μg/ml) have small sizes being in the range of 200-400 nm, have low and moderate polydispersity values and a negative zeta potential of −45 to −33 mV, decreasing with the increasing concentration of HA.

TABLE 4D
Properties of polymeric nanoparticles
comprising HA, CS, and sCT (500 μg/ml)

| | | | | Encapsulated | |
|---|---|---|---|---|---|
| sCT | HA | CL113 | | quantity | % of |
| [mg/ml] | [mg/ml] | [mg/ml] | pH | [μg/ml] | encapsulation |
| 0.500 | 0.714 | 0.284 | 6.07 | 29.73 ± 36.10 | 5.94 ± 7.21 |
| 0.500 | 0.833 | 0.167 | 6.03 | 3.77 ± 3.85 | 0.75 ± 0.77 |
| 0.500 | 0.909 | 0.091 | 6.01 | 0.00 ± 0.00 | 0.00 ± 0.00 |

| | | | Mean | | Zeta |
|---|---|---|---|---|---|
| sCT | HA | CL113 | particle | Polydispersity | potential |
| [mg/ml] | [mg/ml] | [mg/ml] | size [nm] | index | [mV] |
| 0.500 | 0.714 | 0.284 | 216 ± 35 | 0.163 ± 0.047 | −22.5 ± 1.4 |
| 0.500 | 0.833 | 0.167 | 241 ± 31 | 0.170 ± 0.042 | −30.9 ± 2.5 |
| 0.500 | 0.909 | 0.091 | 303 ± 48 | 0.204 ± 0.036 | −−34.1 ± 2.2 |

Table 4D shows that HA/CS polymeric nanoparticles with sCT (500 μg/ml) have even smaller sizes (200-300 nm) than those presented in Table 4C, with low polydispersity values and a negative zeta potential between −34 and −22 mV. The surface charge of the particles becomes more negative with the increasing concentration of HA.

TABLE 4E
Properties of polymeric nanoparticles
comprising HA, CS, and sCT (1000 μg/ml)

| | | | | Encapsulated | |
|---|---|---|---|---|---|
| sCT | HA | CL113 | | quantity | % of |
| [mg/ml] | [mg/ml] | [mg/ml] | pH | [μg/ml] | encapsulation |
| 1.000 | 0.714 | 0.284 | 5.47 | 22.9 | 2.29 |
| 1.000 | 0.833 | 0.167 | 5.45 | 25.3 | 2.53 |
| 1.000 | 0.909 | 0.091 | 5.51 | 23.07 ± 2.78 | 2.31 ± 0.28 |

| | | | Mean | | Zeta |
|---|---|---|---|---|---|
| sCT | HA | CL113 | particle | Polydispersity | potential |
| [mg/ml] | [mg/ml] | [mg/ml] | size [nm] | index | [mV] |
| 1.000 | 0.714 | 0.284 | Aggregates | Aggregates | −14.8 |
| 1.000 | 0.833 | 0.167 | Aggregates | Aggregates | −20.3 |
| 1.000 | 0.909 | 0.091 | 291 ± 61 | 0.234 ± 0.051 | −25.0 ± 1.4 |

Table 4E shows that, with the sCT concentration of 1 mg/ml, polymeric nanoparticles only formed for HA concentration of 0.9 and CS concentration of 0.1 mg/ml. For the other HA/CS ratios studied, micron-sized particles (aggregates) were formed. The polymeric nanoparticles had small particle size, moderate polydispersity index value and were negatively charged.

The increase in sCT concentration used to form the peptide-loaded nanoparticles was accompanied by a decrease in pH. The zeta potential of the polymeric nanoparticles became less negative with the increase in sCT concentration, thus indicating the disposition of the sCT on the particle surface. In the case of formulations with the HA/CL113 mass ratio of 2.5, with the increase in sCT concentration the mean particle size did not change significantly, with the exception of the sample with sCT concentration of 1 mg/ml, where an increase in sCT concentration resulted in the formation of micron-sized particulates, probably due to the decrease in zeta potential, which makes particles prone to aggregation.

The stability of formulations was also decreased with the increasing sCT concentration. The mean particle size of formulations with low concentration of sCT (0.1 mg/ml) and particles without sCT does not differ significantly—There is no significant difference in the mean particle size between the particles with low sCT concentration (0.1 mg/ml) and particles without sCT. For samples with HA/CL113 ratio of 5 and 10, a further increase of sCT concentration caused slight reduction in particle size, with the exception of sample with the HA/CL113 mass ratio of 5, where an increase in sCT concentration resulted in the formation of micron-sized particles.

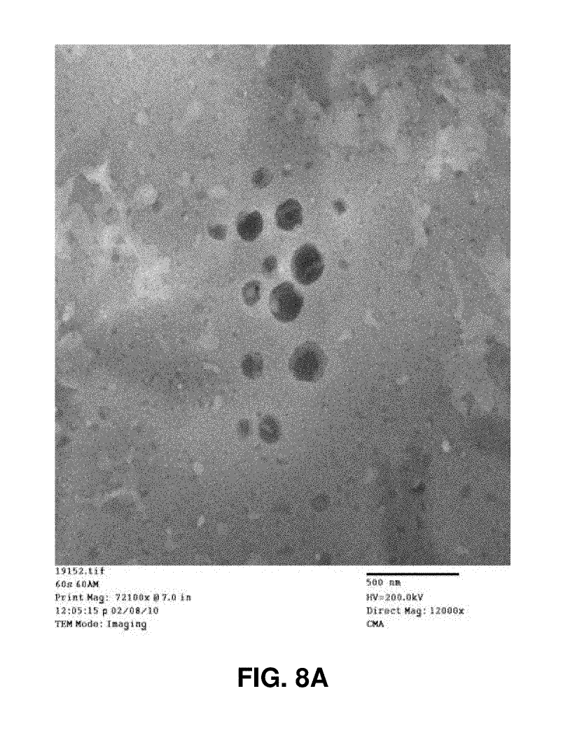

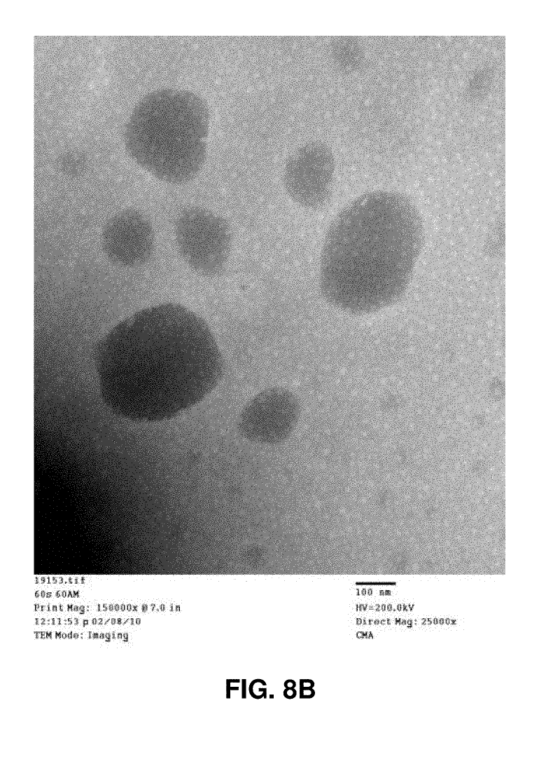

In the case of all polymeric nanoparticles with the same sCT concentration, the increase in the HA/CL113 mass ratio did not change pH significantly, but the zeta potential became more negative (it was decreased by about 10 mV in all cases). For the polymeric nanoparticles with the same sCT concentration the increase in the HA/CL113 mass ratio caused an increase in the particle size and polydispersity index, apart from the formulations with sCT concentration of 1 mg/ml, in which case for lower HA/CL113 mass ratios micron-sized particles were formed. The highest percentage of sCT (loading efficiency) was incorporated by polymeric nanoparticles with sCT concentration 0.1 mg/ml and HA/CL113 ratio of 2.5, but in terms of the encapsulated quantity of sCT probably the formulation with sCT concentration of 1 mg/ml and HA/CL113 ratio of 10 had the best parameters. A TEM photomicrograph of a polymeric nanoparticle comprising hyaluronic acid, chitosan, and sCT (where sCT content is 0.09mg/mg of powder) is presented in FIGS. 8A-B.

Physical parameters of optimised HA/CL113 nanoparticles loaded with sCT are presented in Table 4F

TABLE 4F
Properties of polymeric nanoparticles comprising HA, CS, and sCT

| | Initial | | | | | | |
|---|---|---|---|---|---|---|---|
| | sCT | HA | CL113 | Association | | | |
| | concentration | concentration | concentration | efficiency | Particle | Polydispersity | Zeta potential |
| Sample | [mg/ml] | [mg/ml] | [mg/ml] | [%] | size [nm] | index | [mV] |
| F1 | 1 | 0.909 | 0.091 | 87.4 ± 6.4 | 177 ± 14 | 0.150 ± 0.047 | −29.2 ± 1.1 |
| F2 | 0.5 | 0.909 | 0.091 | 95.3 ± 15.6 | 158 ± 18 | 0.211 ± 0.067 | −45.9 ± 2.3 |
| F3 | 0.35 | 0.909 | 0.091 | 89.4 ± 11.8 | 168 ± 19 | 0.221 ± 0.070 | −54.7 ± 3.8 |
| F4 | 0.35 | 0.833 | 0.167 | 92.1 ± 17.5 | 148 ± 4 | 0.162 ± 0.052 | −37.9 ± 2.2 |
| F5 | 0.2 | 0.833 | 0.167 | 90.6 ± 9.2 | 172 ± 28 | 0.135 ± 0.033 | −44.2 ± 5.4 |
| F6 | 0.2 | 0.714 | 0.286 | 82.8 ± 8.4 | 182 ± 28 | 0.109 ± 0.062 | −25.6 ± 2.2 |
| F7 | 0.35 | 0.714 | 0.286 | 82.0 ± 9.1 | 229 ± 14 | 0.050 ± 0.042 | −19.3 ± 1.8 |
| F8 | 0.5 | 0.833 | 0.167 | 92.5 ± 7.2 | 161 ± 18 | 0.082 ± 0.036 | −30.0 ± 4.0 |

### Example 5

### To Test the Bioactivity of sCT in a Polymeric Nanoparticle Comprising of Hyaluronic Acid and Chitosan on T47D Cell Line

The rationale was to show that the entrapped sCT in the HA/CS nanoparticles was still capable of in vitro bioactivity upon release. This would prove that the formulation process is not damaging to the labile peptide and would provide data that would permit in vivo studies.

T47D cells (American Type Culture Collection (ATCC)) are a human epithelial breast carcinoma cell line enriched with calcitonin receptors. The bioactivity of sCT in a polymeric nanoparticle comprising of hyaluronic acid and chitosan were assessed and compared to native sCT using this cell line.

sCT (calcitonin acetate) was purchased from Polypeptide Laboratories (Denmark) AB. Parameter Cyclic AMP Assay kit (R&D systems #KGE002). All other reagents and chemicals were of cell culture grade. T47D cells were maintained in RPMI 1640 medium supplemented with 10% Foetal Calf Serum, 25 mM HEPES and L-Glutamine, 0.2 units/ml bovine insulin, 1% antibiotics and 1% non-essential amino acids.

Preparation of Treatments: A known concentration of polymeric nanoparticles comprising hyaluronic acid, chitosan, and sCT were placed into RPMI 1640 medium (no serum) with 25 mM HEPES and L-Glutamine and 0.2M 3-isobutyl-1-methyl-xanthine (IBMX). The solution was placed on a magnetic stirrer at 4° C. for 8 hours to ensure the nanoparticles were dissolved and sCT was released from the nanoparticle.

T47D cells were seeded onto 24 well plates at a density of 1×10⁶ cells per well and incubate in 95% air and 5% CO₂ at 37° C. for 2 days. Media was removed and cells washed in serum free media (SFM). Cells were incubated in SFM supplemented with (IBMX, 0.2 mM) at 37° C. for one hour. After one hour media was removed and treatments were added including a positive control of 1×10⁻⁵M Forskolin in SFM and negative control of SFM only. Treatments were made up in SFM with IBMX. Treatments and controls were incubated for 15 minutes and then removed. Cells were removed by adding 0.5 ml of trypsin to each well, incubated at 37° C. until cells have lifted off. 1 mL of media with serum was added to each well to deactivate trypsin. Cells were removed from each well and transferred into an Eppendorf and centrifuged for 10 mins at 10 000 rpm at 4° C. Supernatant was removed and the pellet was washed 3 times using cold PBS. Cells were resuspended in lysis buffer (from R&D cAMP kit) to a concentration of 1×10⁷ cells/mL and stored at −20° C. Freeze/thaw cycle was repeated once to ensure cell lysis. The supernatant was assayed immediately using the cAMP ELISA kit.

The intracellular cAMP generating activities of polymeric nanoparticles comprising hyaluronic acid (HA), chitosan (CS), and salmon calcitonin (sCT) (complexes I, II & III) were compared to native sCT (represents100% bioactivity) using a cAMP assay on T47D cells (FIG. 5). Sample IV represents the polymeric nanoparticle without sCT. All complexes were bioactive where complex I retained 63%, II (88%) and III (78%) of the bioactivity compared to unmodified sCT. For the following experiments sCT:HA complex II was selected.

Referring to FIG. 5, it can be seen that sCT (1×10−8M) represents 100% bioactivity. The same amount of sCT (1×10−8M) was present in each sample except for sample IV (trehalose). In summary, the sCT released from the nanoparticles was comparably bioactive to free unformulated sCT; therefore the formulation process is gentle and results in the release of peptide material that remains bioactive.

### Example 6

### Effects of Polymeric Nanoparticles Comprising Hyaluronic Acid, Chitosan, and Salmon Calcitonin on NR4A1, NR4A2, NR4A3 and MMP Expression in Cytokine-Treated SW1353 Human Chondrosarcoma Cells

Members of the NR4A orphan nuclear receptor subfamily (NR4A1/Nur77, NR4A2/NURR-1, NR4A3/NOR-1) are emerging as key regulators of cytokine and growth factor action in arthritis. The transcription of these NR4A genes is rapidly activated by inflammatory mediators. NR4A subfamily members are aberrantly expressed in inflamed human synovial tissue, atherosclerotic lesions, and psoratic skin; compared with normal tissue. Reports also have identified the role of NR4A receptors as effector molecules of cytokine signalling in other cell types including macrophage cells. In activated cells, NR4A receptors are rapidly induced as transcriptional mediators of inflammatory signals making NR4As a potential target for therapeutic intervention. In addition, matrix metalloproteases (MMPs) are a critical set of proteases that are synthesized by chondrocytes and synovial tissue and they contribute to irreversible degradation of joint components. Since these enzymes are located downstream of NR4A2 signalling, the latter are potentially important modulators of MMPs in arthritis. Thus, NR4A2 is a potential therapeutic target in arthritis and elucidating the mechanism in regulating expression of this receptor may lead to the development of novel strategies to suppress cartilage destruction. sCT with mixtures of HA and chitosan was assessed on NR4A and MMP induced human chondrocyte cell line SW1353 to assess it as a potential therapeutic for targeting these biomarkers in inflammatory diseases.

sCT (calcitonin acetate) was purchased from Polypeptide Laboratories (Denmark) AB. All other reagents and chemicals were of analytical and cell culture grade.

Cell assay: SW1353 human chondrocytes (ATCC) were maintained in RPMI 1640 medium supplemented with 10% Foetal Calf Serum, L-Glutamine and 1% antibiotics. SW1353 cells were seeded 2.5×10⁵ cells per well into 6-well plates. After 24 hours media was replaced with serum free media (SFM) and incubated over night. sCT (10 μM) with either PGE₂(1 μM) or IL-1β (10 ng/ml) were added to cells in SFM for 1 hour. Dexamethasone (Dex, 10 μM) was the positive control for the down-regulation of NR4A2. Untreated controls were treated in an identical manner, but with serum-free medium alone. RNA was isolated from SW1353 cells.

RNA Extraction: Media was removed from the cells and 1 mL of TRIzol® (Invitrogen) was added to each well. Cells were homogenised by pipetting the added TRIzol® up and down for 8-10 times. Cells were placed in labelled eppi and frozen at −80 C until required. Cells were thawed and left at room temperature for 15 minutes. 200 μL of chloroform was added and mixed well and incubate at room temperature for 15 minutes. Samples were centrifuged at 13,000 rpm for 15 minutes at 4° C. The top aqueous layer was removed and placed in a clean eppi—being careful not to remove any of the white or pink layers. 0.75 volumes of isopropanol was added and mixed well. Samples were incubated at room temperature for 15 minutes. Centrifuged at 13,000 rpm for 10 minutes at 4° C. Supernatant was removed and washed pellet with 75% RNA grade ethanol. Centrifuged samples at 8,000 rpm for 5 mins at 4° C. Ethanol was removed and pellet was left to dry. Pellet was then resuspended in an appropriate volume of RNASE/DNASE free water and RNA in each sample was quantified at A₂₆₀.

cDNA Synthesis: RT (Real Time) Reaction was setup as follows in a PCR eppendorf: 2 μg RNA, 1 μL oligo dT, 1 μL dNTP Mix (10 mM) all to a final volume of 10 μL with H₂0.

‘RT’ program on PCR machine: −65° C., 5 min., pause at 4° C. Add 8 μL of mix to each tube (also prepare 1 less MMLV—negative control):

Mix to be added to each reaction tube is 4 μL of 5× 1st Strand Buffer, 2 μL of 0.1 M DTT, 1 μL of RNase Out and 1 μL MMLV. Taq Cat# 10342-020 (this contains 0.1 M DTT, 5× 1st S. B.). NR4A and MMP1, 3, 9 and 13 levels were measured by qRT-PCR and expression levels were normalized to GAPDH levels.

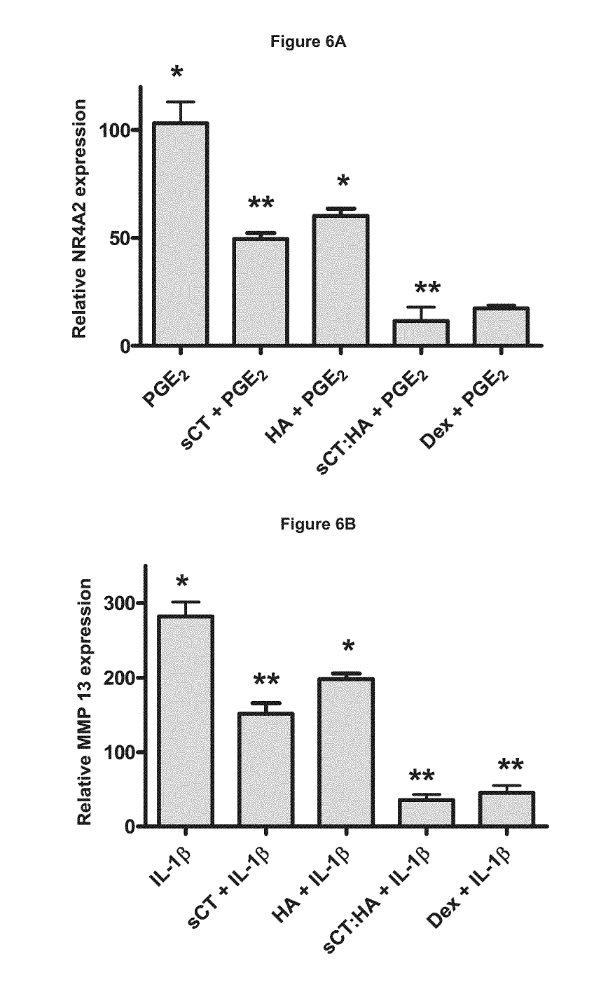

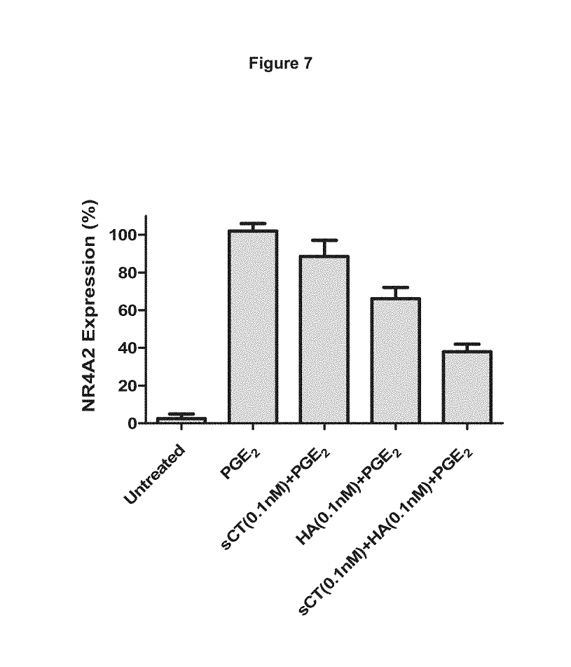

Referring to FIG. 6A, PGE₂ rapidly and potently induced NR4A2 mRNA in human chondrocyte cells (SW1353) after one hour. SW1353 cells were treated with sCT alone or polymeric nanoparticles comprising hyaluronic acid, chitosan, and sCT and PGE₂ for 1 hour. Total RNA was extracted and assayed for NR4A2 mRNA levels by quantitative RT-PCR. Both sCT alone, HA alone, and polymeric nanoparticles comprising hyaluronic acid, chitosan, and sCT inhibited PGE₂ induced NR4A2 transcript levels by 49%, 41% and 91% respectively, with the mixtures containing the HA component of the nanoparticles quite potent when compared to dexamethasone. Similar results were also obtained where the nanoparticle HA component in a mixture format with sCT down regulated PGE₂ induced NR4A1 (87%) and NR4A3 (82%) transcript levels (results not shown). Importantly, in each case the combination of sCT with HA in gave superior reduction in expression of the inflammatory biomarkers than either agent alone at the same concentrations.

These studies indicate that sCT is involved in the inactivation of the NR4A receptor signalling pathways. Its potency is enhanced when mixed specifically with HA.

Effects of sCT and the nanoparticle on MMP mRNA levels were also investigated. IL-1β potently induced MMPs in SW1353. Both sCT, HA, and the polymeric nanoparticle down regulated MMP 13 by 47%, 31% and 88% (FIG. 6B). MMPs 1 and 3 were also down regulated. However no effect was observed for MMPs 2 and 9 (results not shown). Dexamethasone, which is known to down regulate NR4A2 and is used for the treatment of rheumatoid arthritis, was used as a positive control in all experiments. MMPs are downstream biomarkers of inflammation from NR4A pathway, so the data confirms that the novel nanoparticles have anti-inflammatory actions at two different sites on the pathway in these cells.

Synergistic effect of the combination of sCT and HA were investigated on SW1353 cells as described on Line 16 page 17—Cell assay. The results clearly show that combinations of HA with sCT give superior effects than either agent alone at the same concentration.

### Example 7

### The In Vitro Release Properties of Polymeric Nanoparticles Comprising Hyaluronic Acid, Chitosan, and sCT, Carried Out in a PBS Solution

1 ml of polymeric nanoparticles (equivalent to 1.2 mg of dry nanoparticles for A, 1.1 mg for B, 1.2 mg for C and 1.4 mg for D) was suspended in 9 ml of PBS forming 10 ml of the release studies system. 2.5 ml of the suspension was withdrawn at the first time point and the release medium was separated from the polymeric nanoparticles using filtration/centrifugation. The retained polymeric nanoparticles were re-suspended up to 2.5 ml with PBS and the studies continued. At each time point, the whole 2.5 ml was separated from the release medium and then reconstituted with PBS up to 2.5 ml. The amount of sCT released at each time point was assayed by HPLC.

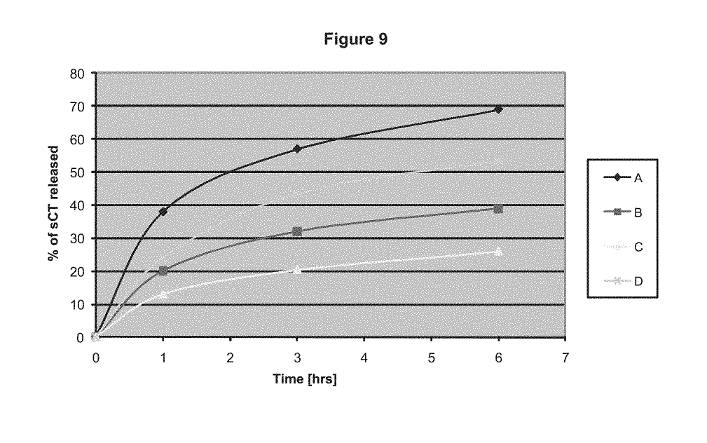

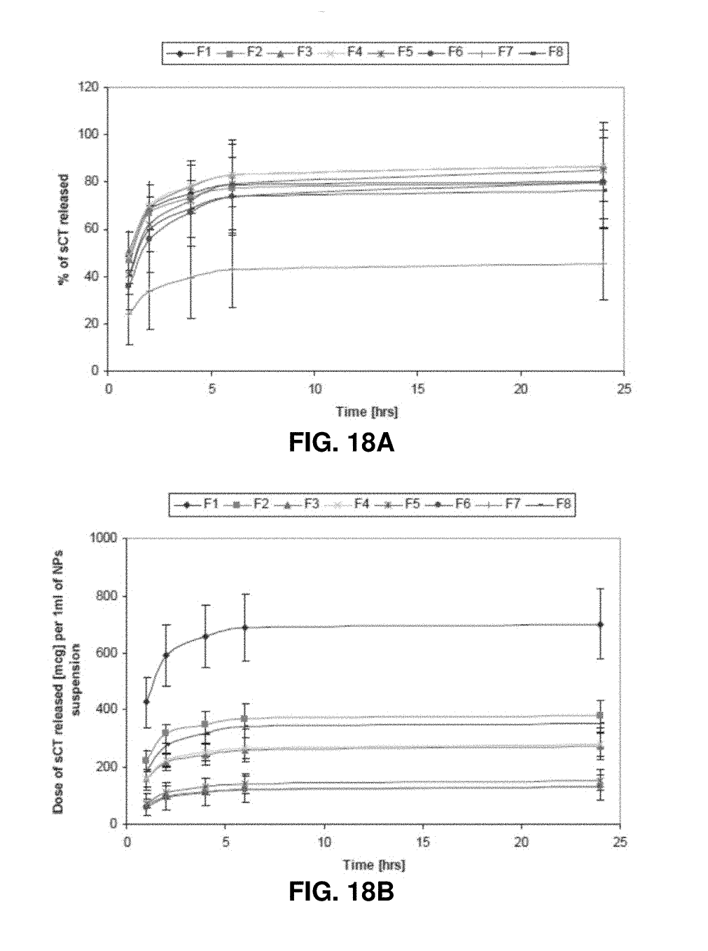

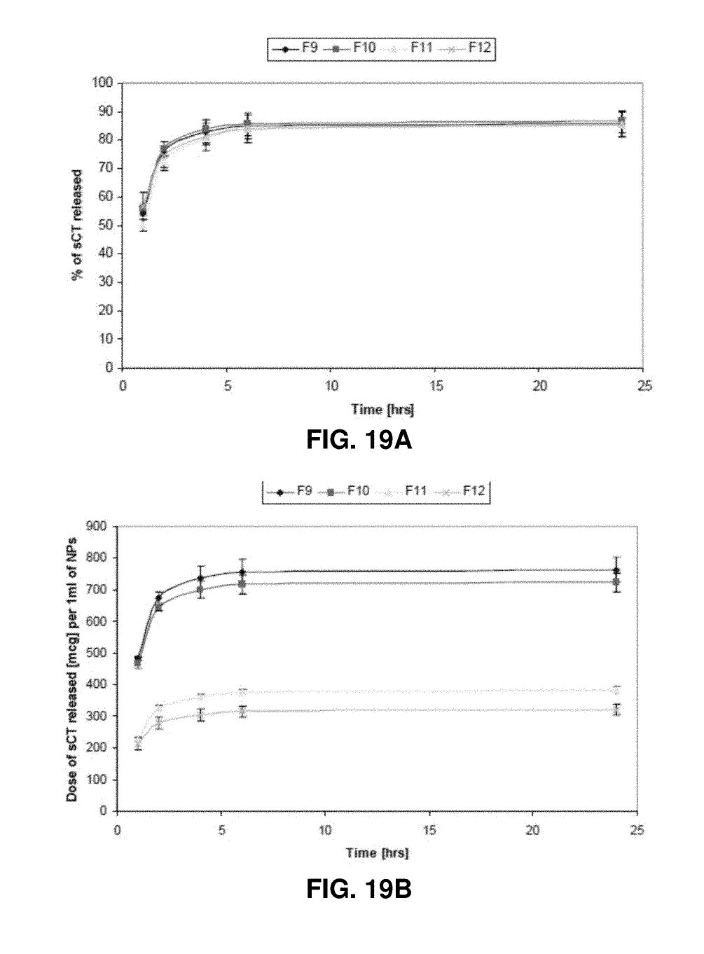

Referring to FIGS. 9 and 18A-B, it would appear that chitosan decreases the rate of sCT released.

### Example 8

### Polymeric Nanoparticles Comprising Hyaluronic Acid, Protamine, and Salmon Calcitonin

TABLE 5A
The physicochemical properties and sCT association efficiencies of polymeric
nanoparticles comprising hyaluronic acid, protamine, and salmon calcitonin

| | | | | | Zeta | |
|---|---|---|---|---|---|---|
| sCT | uHA | Protamine | Mean particle | Polydispersity | potential | Efficiency |
| [mg/ml] | [mg/ml] | [mg/ml] | size [nm] | index | [mV] | [%] |
| 0.1 | 0.714 | 0.286 | 160 ± 1 | 0.105 ± 0.010 | −35.3 ± 0.6 | 91 |
| 0.1 | 0.714 | 0.343 | 165 ± 1 | 0.112 ± 0.013 | −33.7 ± 0.6 | 85 |

TABLE 5B
The physicochemical properties and sCT association efficiencies of polymeric
nanoparticles comprising hyaluronic acid, protamine, and salmon calcitonin

| | Initial | | | | | | |
|---|---|---|---|---|---|---|---|
| | sCT | HA | P | Association | | | |
| | concentration | concentration | concentration | efficiency | Particle | Polydispersity | Zeta potential |
| Sample | [mg/ml] | [mg/ml] | [mg/ml] | [%] | size [nm] | index | [mV] |
| F9 | 1.0 | 1.429 | 0.343 | 88.9 ± 1.8 | 282 ± 21 | 0.243 ± 0.021 | −37.7 ± 3.8 |
| F10 | 1.0 | 1.429 | 0.457 | 83.5 ± 3.5 | 342 ± 70 | 0.303 ± 0.100 | −25.4 ± 2.8 |
| F11 | 0.5 | 1.429 | 0.457 | 89.8 ± 2.0 | 246 ± 25 | 0.209 ± 0.064 | −37.1 ± 1.7 |
| F12 | 0.5 | 1.429 | 0.686 | 75.1 ± 3.5 | 503 ± 133 | 0.498 ± 0.134 | −26.9 ± 3.5 |

The examples below refer to preparation of empty polymeric nanoparticles. Examples 9-15 describe optimisation of the method that is the currently the final, most refined technical method that was later utilised in preparation of sCT-loaded HA/chitosan nanoparticles.

### Example 9

### The Influence of Ultrasound on HA

These studies allowed to the optimisation of the molecular weight of HA required for the successful production of HA-based nanoparticles.

Ultrasonication is a simple and efficient method of obtaining polymers of lower molecular weight from the original high molecular weight compounds. Viscosity and GPC results (see FIG. 13) confirmed that the high molecular weight hyaluronate samples were effectively depolymerised even after a relatively short exposure to ultrasounds. The viscosity of the native 0.1% HA solution is about 17 times higher than the viscosity of water at 25° C. Ultrasonication produced a permanent reduction in the viscosity of HA solution. The decrease was very rapid during the first minutes and but after prolonged sonication no marked reduction in viscosity (molecular weight) of HA solution was seen. Ultrasonic treatment also results in the reduction of the weight average molecular weight (Mw) as a result of depolymerisation.

Our results confirmed that the degradation of HA is significantly higher at higher molecular weights. It is generally accepted that mechanical force is the factor causing the depolymerisation of HA during sonication process. Small bubbles are formed in the liquid (cavitation) due to propagation of acoustic energy causing rapid pressure variations. The polymer chains are broken due to the large velocity gradients generated close to collapsing cavitation bubbles. The long chains of the polymer molecules may not be able to follow the flow of the molecules of the solvent, and so the concentrated tension is high enough to break the chain. Miyzaki et al., demonstrated that depolymerisation on sonication is not a thermal effect. They also proved that chemical changes played a minor role. However, the depolymerisation was affected by the dissolved gas. One of the most important factors affecting the cavitation is ultrasonic intensity (the acoustic pressure amplitude). The cavitation bubbles cannot be formed under a certain pressure variation. The increase of intensity results in the increase of the number of bubbles as well as their maximum size, and thus strengthens the cavitation activity. In consequence the rate of depolymerisation increases and the degree of depolymerisation is enhanced. The characteristics of mechanical depolymerisation are non-random kinetics and a limiting molecular weight.

### Example 10

### Impact of Polymer Mixing Ratio

These studies allowed systematic investigations of the properties of HA-based nanoparticles depending on the polymer mixing (volume or mass or charge) ratio used. The polymer mixing ratio determines the size and zeta potential of the particulates, thus in turn impacting on incorporation of an active molecule.

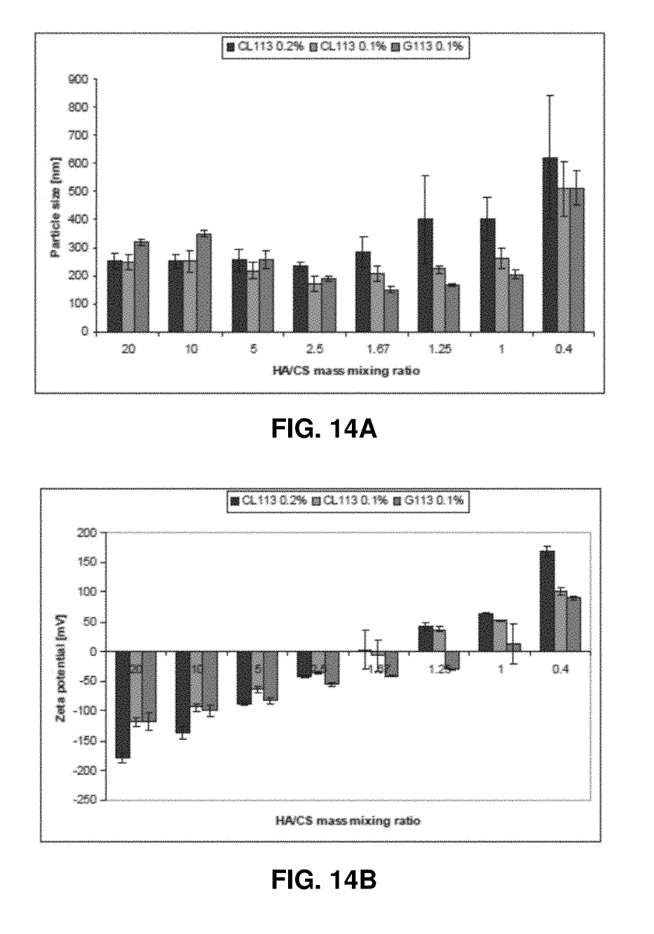

When a large excess (in terms of charge mixing ratio) of one of the polymers was used, the sample had the appearance of a solution, what was confirmed by transmittance measurement (transmittance values at 500 nm were close to 100%). When the quantities of polymers became more stoichiometric, the system became at first opalescent, then turbid, and finally when the quantities of polymers were stoichiometric and the opposite charges were neutralised, aggregation and phase separation occurred. The lower the excess of one of the polymers (in terms of charge mixing ratio), the lower was the viscosity (see FIG. 14A). The zeta potential values increased from high negative values to high positive values with a decrease in the HA/chitosan ratio and the surface charge underwent an inversion when the charge mixing ratio was close to 1. Apart from the formulations with the amounts of polymers close to stoichiometric all formulations had the absolute value of zeta potential above 30 mV, which indicates their good stability (see FIG. 14B).

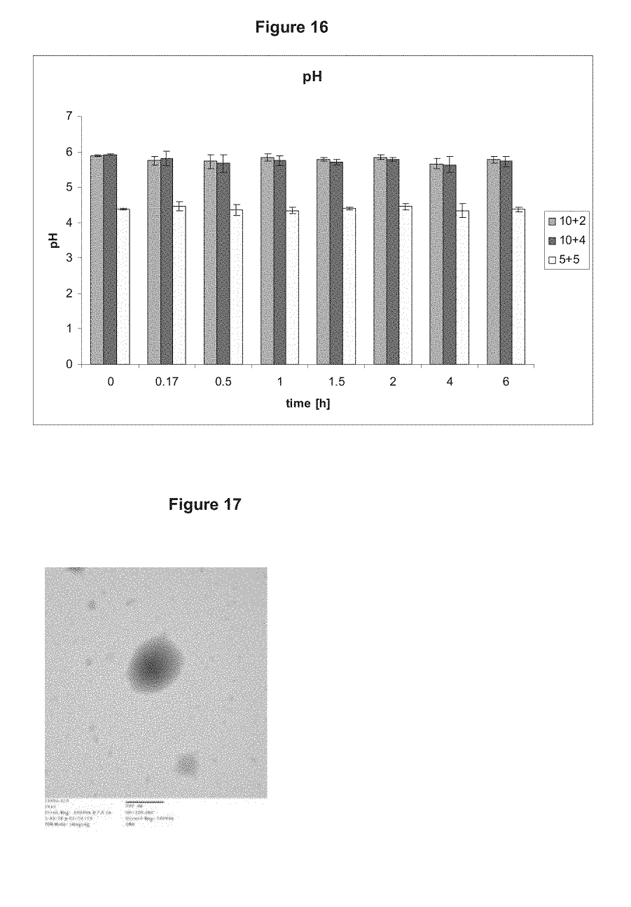

pH was reduced slightly when the HA/chitosan mixing ratio decreased in case of anionic particles, although the changes were not always significant. The value is below 6 in most formulations (see FIG. 16). With further increase in the chitosan content, it can be observed that pH was reduced rapidly to values around 4.5 and lower. The zone of the rapid reduction of pH corresponds to the zone of zeta potential inversion. The pH of liquid (continuous phase) may impact on the physical stability of nanoparticles.

Generally for anionic particles the particle size at first decreased slightly or did not change significantly with the decreasing HA/CL113 ratio, and when the polymers mixing ratio was close to the neutralisation point, the particle size increased and micron-sized particles were formed. The particle size of cationic particles increased with the increase in chitosan content.

Similar tendency may be observed for PDI values: generally they decrease with increasing content of chitosan (but in case of formulations with high excess of HA the changes are not significant), then reach the low just before the inversion of particle charge, and then increase again with further increase of chitosan content—in the zone of charge neutralisation however the tendency may be disrupted due to the formation of aggregates.

### Example 11

### Impact of Total Polymer Concentration

These studies allowed the investigation of the impact of HA solution concentration on the properties of HA-based nanoparticles formed. More concentrated solutions had greater viscosities affecting the properties of nanoparticles.

Generally, the amount of particles formed was greater for the higher polymer concentration (transmittance measurements and visual assessment the 0.2% w/v formulations were more turbid compared to 0.1% w/v). It could be observed that when the excess of one of the polymers was used, the viscosity of 0.2% w/v nanoparticle suspension was higher than the viscosity of 0.1% suspension, and between the mass ratios of HA/CL113 of 10:4 and 5:5 the viscosities were similar. For the negatively charged nanoparticles, when a large excess of HA is used, the particle size of 0.1 and 0.2% w/v formulations did not differ significantly, however with a further decrease in the HA/CL113 ratio, the 0.1% w/v nanoparticles became significantly smaller compared to the 0.2% w/v nanoparticles. It can be seen that for the 0.2% w/v concentration the particles had greater surface charge, when compared to those of 0.1% w/v.

Generally, an increase in the total polymer concentration to 0.2% w/v led to the formation of larger particles compared to the 0.1% w/v formulations (see FIG. 14A). FIGS. 14A-B contain data for both 0.1 and 0.2% w/v HA concentrations and illustrates the polymer mixing ratio, total polymer concentration and impact of the salt type.

### Example 12

### Impact of Chitosan Salt and Molecular Weight

These studies allowed the investigation of the impact of the type of chitosan salt on the properties of HA-based nanoparticles. It is known that the different salts may have different solubilities, molecular weight and charge affecting the properties of nanoparticles.

Large, micron-sized particles (agglomerates/aggregates) were formed when high molecular weight salts of chitosan were used (CL213 and G213). In those cases, the samples were allowed to settle overnight in order to remove aggregated particles. Even after sedimentation of aggregates the transmittance of CL213 NPs was smaller than that of CL113 NPs, which did not form aggregates. This may suggest that when longer chitosan chains were used, more particles were formed as more amino groups were available to interact with the carboxyl groups of HA and the interacting polymer chains precipitated as nanoparticles or micron-sized aggregates.

The transmittance values were similar for the nanoparticles made of HA and either G213 or G113 or the differences between G213 and G113 were smaller compared to the chitosan chloride pair, and for cationic G213 NPs the transmittance was even smaller than for G113 NPs.

When the HA/CL weight mixing ratio was close to 5:3 or 5:4, the viscosity value reached the low of 0.89-91 Pa*s, which is similar to the viscosity of water at 25° C. Interestingly, for chitosan glutamate NPs the minimum is shifted to the HA/G weight mixing ratio of 5:5.

Even after sedimentation of aggregates, the particle size and polydispersity indices of CL213 and G213 NPs were higher than CL113 and G113 NPs, respectively.

For chitosan chloride nanoparticles the charge-mixing ratio did not correspond to the weight-mixing ratio. When equal amounts of polymers were mixed, the particles were positively charged, which suggests that the chitosan charge dominates over HA charge and chitosan chloride has a greater charge density than HA. That difference in charge density of both polymers influences also the zone of the formation of nanoparticles. For example, when the weight mixing ratio for HA/CL113 was 10:4, a turbid nanoparticle suspension was formed (small excess of HA), and when the ratio was 4:10, the sample had an appearance of a transparent solution (large excess of chitosan).

Comparing glutamate and chloride NPs, it was observed that the glutamate nanos were more negatively charged. The zone of charge neutralisation was shifted to the formulation with the weight mixing ratio for glutamate NPs, so the weight and charge mixing ratio are similar.

The mean particle size and zeta potential values for the different samples are presented in FIGS. 14A and 14B.

### Example 13

### Impact of HA Molecular Weight

HA molecular weight was investigated systematically to elucidate its impact on the properties of HA-based nanoparticles.

When HA solution was not sonicated prior to the formation of nanoparticles, micron-sized particles were formed after mixing with chitosan solution. When HA solution was treated with ultrasounds, for sonication periods of 30 minutes and longer, it was possible to obtain nanoparticles without the tendency to sediment. Such a long ultrasonication time enables to obtain HA with the molecular weight much lower than the non-sonicated HA (1700 kDa and 450 kDa, respectively). A further increase in the sonication time led to the reduction in the molecular weight of HA.

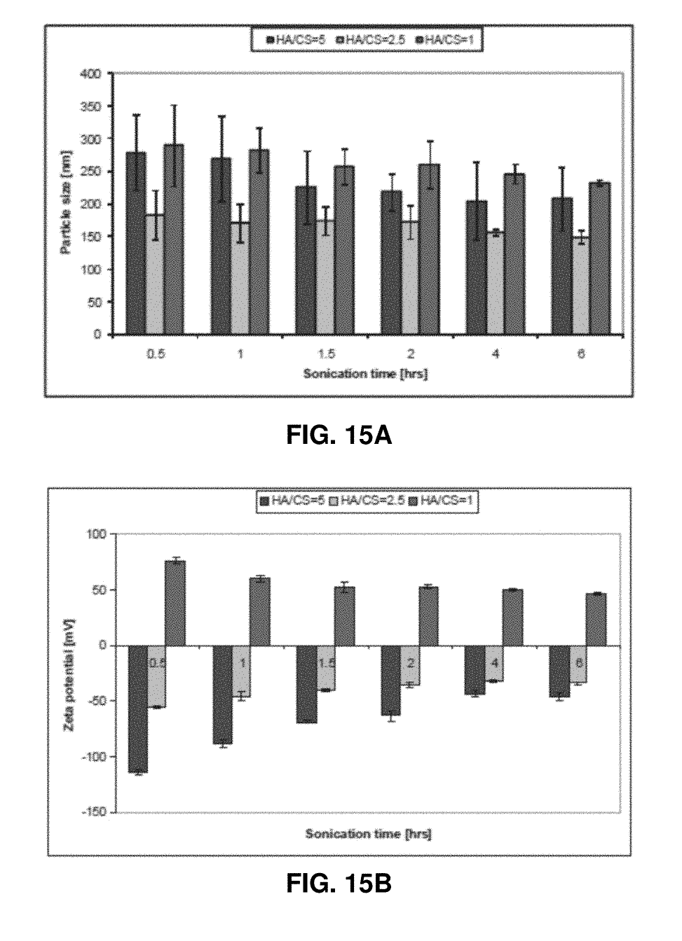

As mentioned earlier, when HA was not sonicated or the sonication of HA prior to the preparation of NPs was 10 minutes, the formation of nanoparticles was accompanied by the formation of micron-sized particles. For all the HA/CL113 mass ratios tested (10:2 (illustrated as “5”), 10:4 (illustrated as “2.5”), 5:5 (illustrated as “1”)) the sonication of HA resulted not only in the lack of micron-sized aggregates, but in a decrease in particle size as well (see FIG. 15A). The zeta potential values were also affected by the increase in the sonication time and with longer sonication times the surface charge decreased. The reduction in the absolute zeta potential values was more pronounced when HA solutions sonicated for shorter periods were used and can be observed in negatively as well as positively charged nanoparticles obtained (see FIG. 15B). The results are summarised in FIGS. 15A and B.

### Example 14

### The Morphology of the Nanoparticles

The TEM micrograph (see FIG. 17) shows that the particles are roughly spherical, but in some cases deformations, such as non-spherical particles, can be observed. When the HA/CL113 mass mixing ratio was of 2.5, the appearance of the particles was dense and well defined, while the nanoparticles with the HA/CL113 mass mixing ratio of 5 were composed of a relatively dense core surrounded by a less dense and diffused corona. This example shows the physical structure of HA/CL113 nanoparticles.

### Example 15

### pH Titration Studies

The value of zeta potential is related to the stability of colloidal dispersions, e.g. nanoparticles. It indicates the degree of repulsion between adjacent, similarly charged particles in dispersion. High values of zeta potential (either positive or negative) result in electrical stabilization while colloidal systems with low superficial charge have the tendency to coagulate or flocculate. Any change in the pH of the environment may have a significant influence on the stability of nanoparticles. In physiological conditions nanoparticles may be exposed to different range of pH. Thus isoelectric point (the pH at which a particular molecule or surface carries no net electrical charge) is very important property. At a pH near the isoelectric point (±2 pH units) colloids are usually unstable and flocculation is likely to occur. Also, if the molecule encapsulated in the nanoparticles interacts with the polymers by electrostatic forces, it is important that the charge of net charge of particles and the charge of the molecule are opposite. If the particles are anionic (thus the pH of the medium is higher than their isoelectric point), the molecule must be positively charged in order to interact electrostatically with the particles, so the formulation will require pH lower than the isoelectric point of this molecule. A summary of the results is given in the table below:

TABLE 6
The values of isoelectric point for different HA/CS NPs.

| HA | CS | | | |
|---|---|---|---|---|
| concentration | concentration | Type of | HA/CS mass | Isoelectric |
| [mg/ml] | [mg/ml] | CS salt | mixing ratio | point |
| 0.714 | 0.286 | G113 | 2.5 | 2.87 ± 0.09 |
| 1.429 | 0.571 | CL113 | 2.5 | 2.86 ± 0.09 |
| 0.833 | 0.167 | CL113 | 5 | 2.47 ± 0.25 |
| 0.714 | 0.286 | CL113 | 2.5 | 2.83 ± 0.38 |
| 0.5 | 0.5 | CL113 | 1 | 6.98 ± 0.54 |

### Example 16

### K/BxN Mouse Model of Arthritis

The K/BxN mouse model of arthritis was used to confirm our in vitro data and to assess the therapeutic effect (reduction in inflammation in the knee) of intra-articular delivery of the polymeric nanoparticles comprising hyaluronic acid, chitosan, and salmon calcitonin into the knee.

Mice expressing the KRN T cell receptor transgene and the MHC class II molecule A(g7) (K/BxN mice) develop severe inflammatory arthritis, and serum from these mice causes similar arthritis in a wide range of mouse strains, owing to pathogenic autoantibodies to glucose-6-phosphate isomerase (GPI). This model has been useful for the investigation of the development of autoimmunity (K/BxN transgenic mice) and particularly of the mechanisms by which anti-GPI autoantibodies induce joint-specific inflammation (serum transfer model).

Passive K/BxN arthritis is an antibody-induced arthritis that is dependent solely on innate immunity, including complement, neutrophils, mast cells, and macrophages. These mice develop an arthritis that is mediated, and transferable, by circulating antibody against GPI. The K/BxN model has distinguishing features:(i) spontaneous disease (24-48 hrs) (ii) ability to transfer disease to a wide range of recipient strains using serum containing the pathogenic autoantibodies; (iii) robust development of arthritis in 100% of transgenic animals and nearly 100% of wild-type mice that have received K/BxN serum; and (iv) inflammation is confined to the 4 paws.

Intra-articular delivery of sCT:HA NPs: 6-8 week old male BALB/c mice were injected intraperitoneally with 150 μl of serum from K/BxN mice on day 0 to induce arthritis. The arthritis index is assessed on a scale of 0-3 (0=no swelling or erythema, 1=slight swelling or erythema, 2=moderate erythema and swelling in multiple digits or entire paw, 3=pronounced erythema and swelling of entire paw; maximum score of 12/mouse). Mice developed a clinical score >1 in at least one paw before treatment. Anesthetized mice were administered drug treatment by i.a injection. The skin above the knee was shaved, and indicated doses of sCT, dex, sCT:HA NPs (5 μl), were injected intra-articularly into the left knee joint using a Hamilton syringe with a 30-gauge needle. The right knee of each mouse was only injected IA with PBS (internal control). The arthritis index was assessed daily by (i) paw thickness (plethysmometer); (ii) knee diamater (dial calipers) and (iii) all four paws clinically scored using the scoring system developed by Benoist et al.

On day 4 after K/BxN serum transfer, mice were sacrificed, and their hind paws above the knee joint were harvested for histology to assess the effects of drug treatments on inflammation and bone and cartilage degradation. The histology sections were stained with hematoxylin and eosin (H&E) and Saffarin O and scored blindly under 3 different categories (i) inflammation (synovitis infiltration and pannus formation), (ii) cartilage erosion and (iii) bone erosion. RNA was also isolated from both the ankle joint and knee joint tissue to measure expression of NR4A biomarkers.

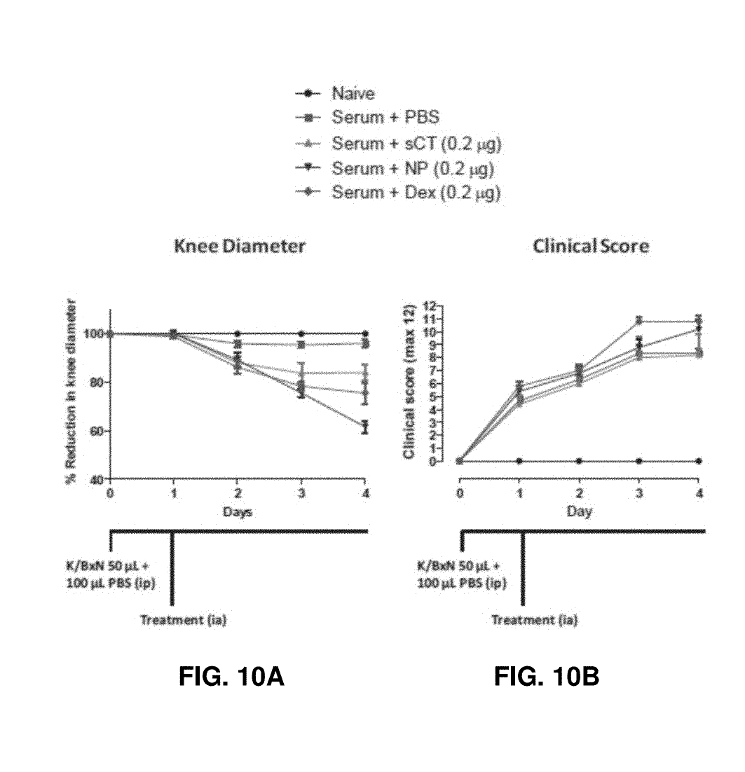

Referring to FIGS. 10A-B, it can be seen that, in the K/BxN mouse model of inflammatory arthritis, a polymeric nanoparticle comprising hyaluronic acid, chitosan, and salmon calcitonin delivered by the IA route reduced knee diameter over time.

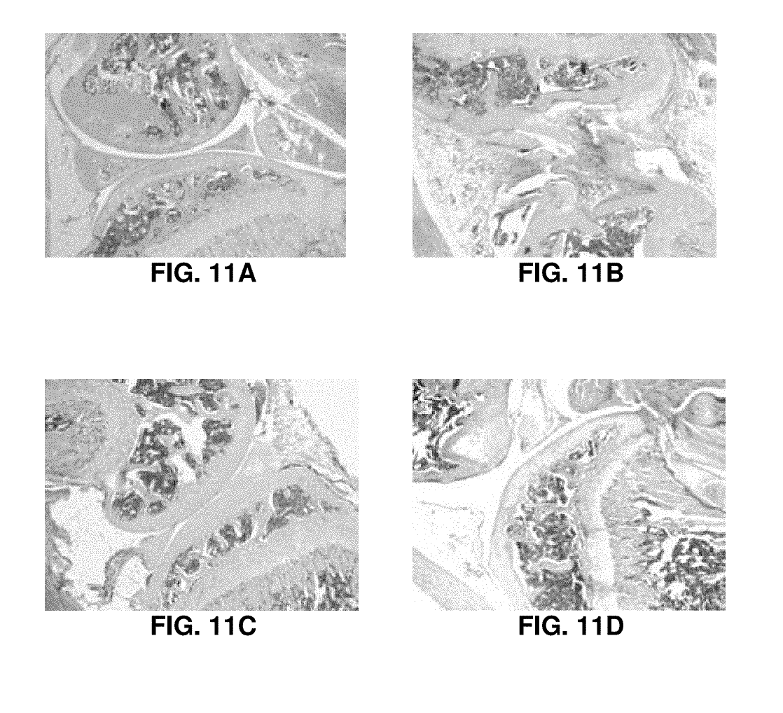

Histology of the knee joint also shows that sCT NP preserves both bone and cartilage and reduces synovitis infiltration damage compared to controls (FIGS. 11A-D and 22A-E).

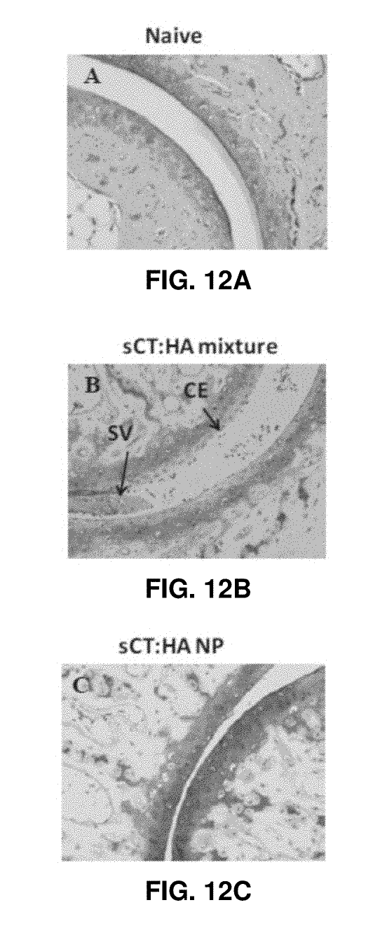

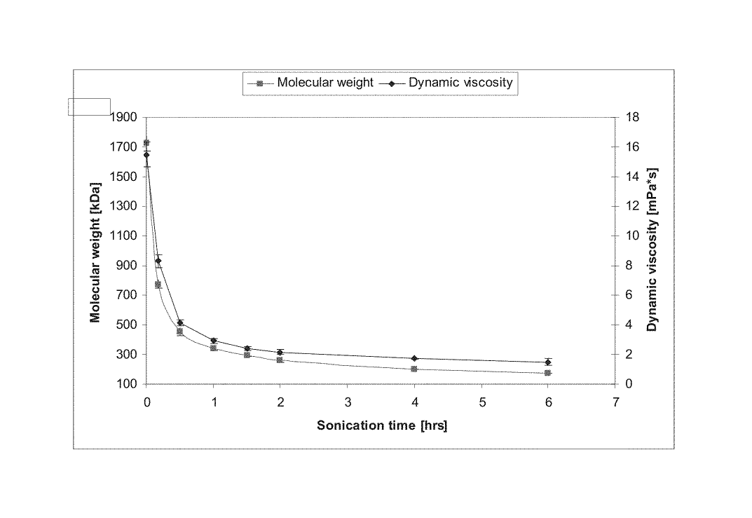

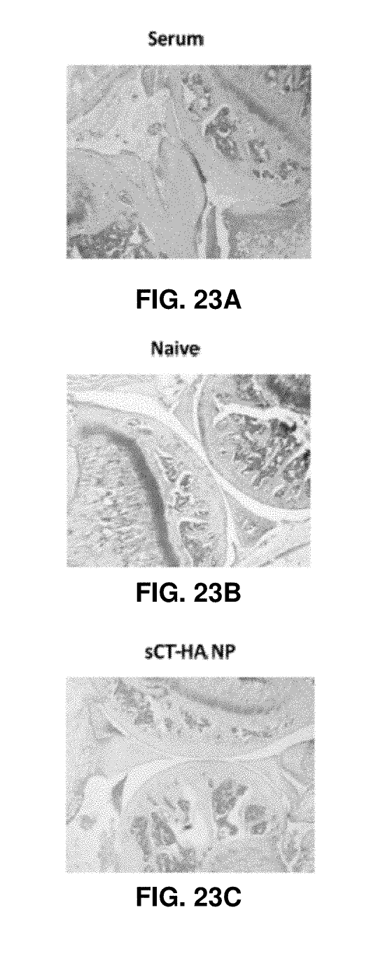

sCT formulated into a NP was superior to free sCT in cartilage preservation showing that formulated into a NP improves the therapeutic effect and sustained release of sCT showing the IA route of delivery has great potential (FIGS. 12 and 23).

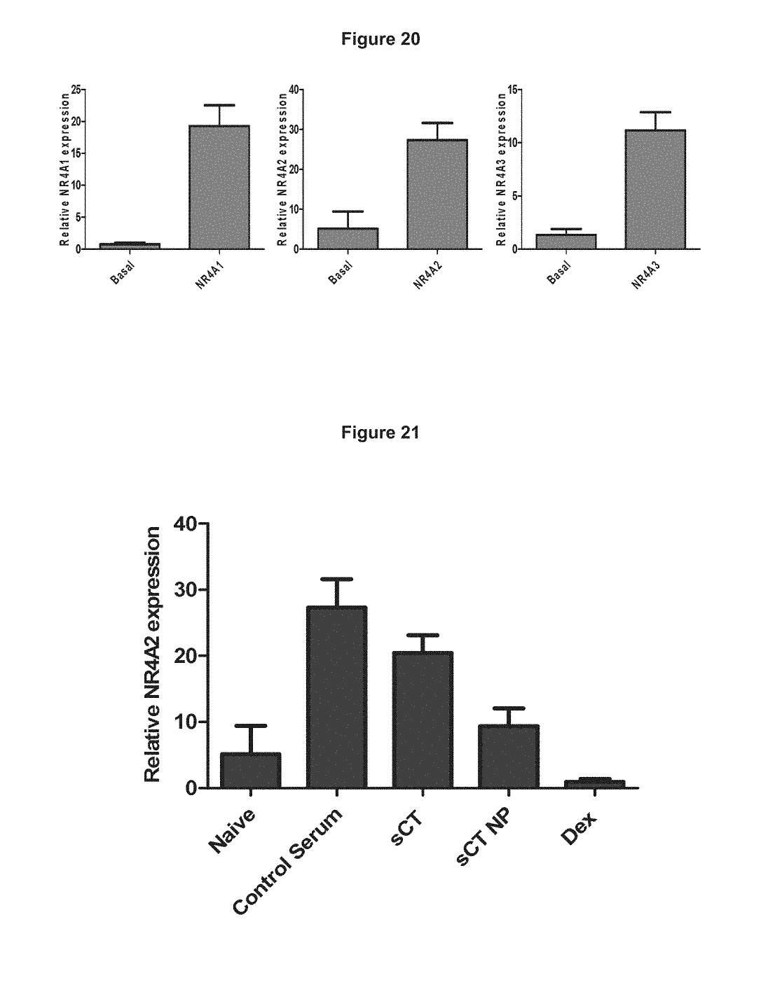

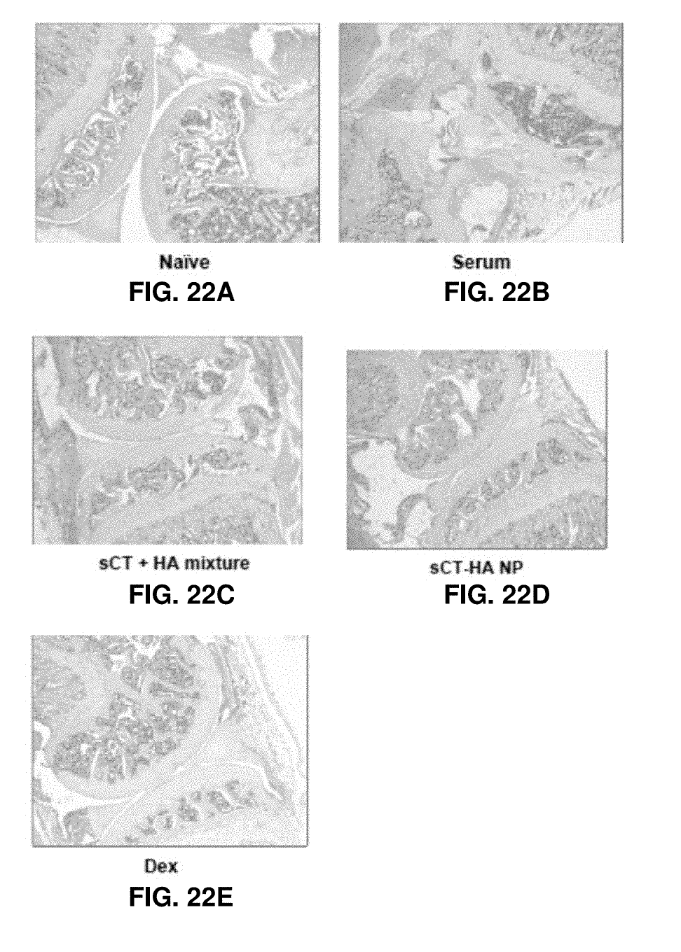

RNA was isolated from the ankle joints of control and ‘inflamed’ K/BxN mice to measure expression of NR4A biomarkers. In K/BxN sera-induced mice with no treatment (therefore showing inflammation), high levels of NR4A1, NR4A2 and NR4A3 were over expressed, compared to naïve mice with no serum transfer (no inflammation (FIG. 20).

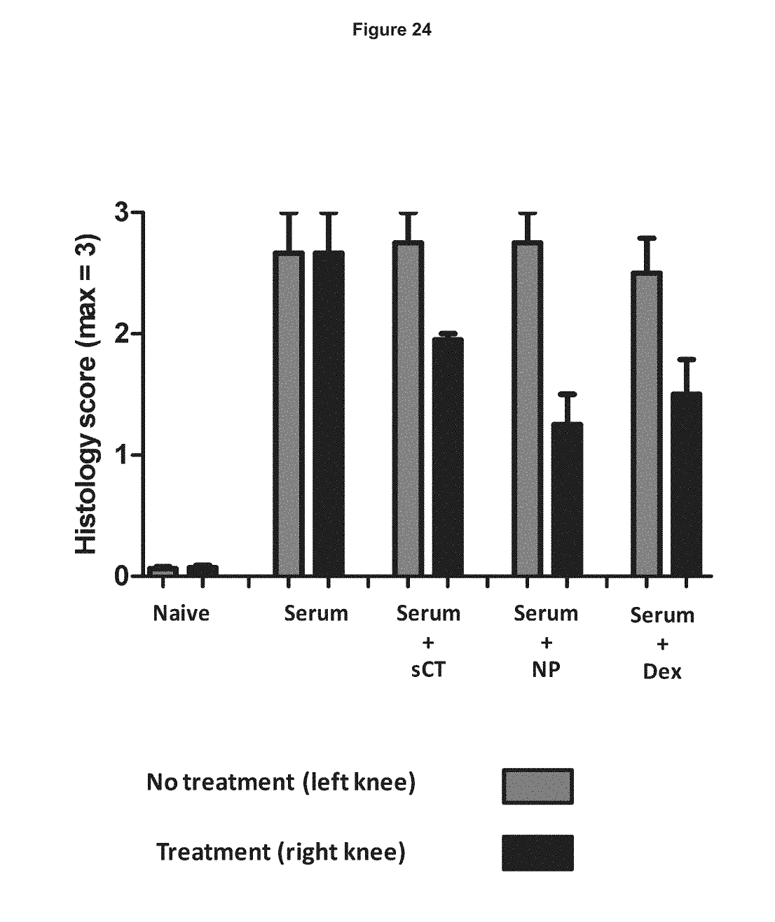

Expression of NR4A2 was measured in all groups (FIG. 21). All anti-inflammatory treatments (sCT:HA mixture, sCT:HA NP and dex) reduced NR4A2 expression, however the effect of sCT:HA NP in reducing NR4A2 expression was superior to ad-mixed sCT:HA in ankles following intra-peritoneal delivery. Histological sections of the knee joint were scored by an observer blinded to the treatment under 3 different criteria (i) inflammation (synovitis infiltration and pannus formation), (ii) cartilage erosion and (iii) bone erosion on a scale of 0-3 (0, none; 1, minimal invasion of meniscus; 2, partial invasion of meniscus; 3, severe invasion of meniscus). All treatments gave a lower histology score, but sCT:HA NP giving the lowest (FIG. 24, intra-articular delivery)

### Example 17

### Stability of HA/CL113 Nanoparticles After Storage at Room Temperature

Table 7 shows the change in physical parameters of the different nanoparticles during 28 days storage at room temperature. Positively charged nanoparticles were the least stable formulations. First signs of sedimentation were visually observed after two weeks of storage, while in the case of negatively charged particles the first signs of sedimentation were noticeable after 3 weeks. After 4 weeks at the bottom of the vial it was possible to observe sediment for all samples. The particle size of positively charged nanoparticles decreased systematically upon 28 days storage from 239 to 152 nm, while the size of negatively charged nanoparticles (HA/CL113 mass mixing ratio of 2.5) systematically increased during storage from 188 to 221 nm.

TABLE 7
Change in physical parameters of the different nanoparticles during 28 days storage at
room temperature.

| | PS of | PS of | PS of | ZP of | ZP of | ZP of |
|---|---|---|---|---|---|---|
| Day | HA/CL113 = 2.5 | HA/CL113 = 5 | HA/CL113 = 1 | HA/CL113 = 2.5 | HA/CL113 = 5 | HA/CL113 = 1 |
| 0 | 188 ± 4 | 221 ± 6 | 239 ± 3 | −36.7 ± 0.9 | −61.8 ± 1.4 | 52.5 ± 1.1 |
| 1 | 193 ± 3 | 221 ± 10 | 215 ± 4 | −35.6 ± 1.4 | −58.0 ± 2.3 | 50.7 ± 0.9 |
| 2 | 201 ± 4 | 239 ± 13 | 206 ± 3 | −36.2 ± 0.2 | −55.3 ± 0.6 | 51.1 ± 1.1 |
| 3 | 201 ± 2 | 247 ± 15 | 200 ± 3 | −36.2 ± 0.7 | −53.4 ± 3.1 | 50.2 ± 1.1 |
| 4 | 202 ± 3 | 217 ± 6 | 197 ± 3 | −38.2 ± 0.3 | −57.6 ± 0.7 | 51.5 ± 1.1 |
| 6 | 206 ± 3 | 215 ± 8 | 190 ± 3 | −37.7 ± 0.6 | −53.4 ± 4.2 | 50.3 ± 1.2 |
| 8 | 207 ± 3 | 217 ± 8 | 186 ± 2 | −38.6 ± 0.5 | −55.5 ± 3.6 | 50.6 ± 1.3 |
| 11 | 211 ± 4 | 218 ± 8 | 181 ± 2 | −38.7 ± 1.8 | −59.0 ± 2.5 | 51.7 ± 0.5 |
| 14 | 211 ± 4 | 224 ± 11 | 176 ± 2 | −39.5 ± 0.8 | −59.5 ± 2.0 | 51.6 ± 0.5 |
| 21 | 213 ± 5 | 236 ± 6 | 164 ± 3 | −40.4 ± 1.0 | −63.5 ± 1.9 | 51.0 ± 1.0 |
| 28 | 221 ± 3 | 228 ± 4 | 152 ± 2 | −37.0 ± 1.6 | −56.1 ± 4.3 | 47.1 ± 1.0 |

PS: Particle size [nm]
PDI: Polydispersity index
ZP: Zeta potential [mV]
Total polymer concentration was of 0.1%.

This example shows that the nanoparticles were stable in suspensions for at least 2 weeks.

Materials and Methods

Materials

Hyaluronic acid sodium salt from Streptococcus equi sp. was purchased from Sigma, USA or NovaMatrix, Norway. Ultrapure chitosan salts, e.g. chloride (Protasan UP CL113) were obtained from NovaMatrix, Norway. sCT (calcitonin acetate) was purchased from Polypeptide Laboratories (Denmark) AB. All other reagents, chemicals and solvents were of analytical grade. The sCT was obtained from PolyPeptide Pharma (Copenhagen).

Ultrasonication

Sodium hyaluronate (HA) solution prepared at concentration 0.1% or 0.2% w/v in deionised water was sonicated with the aid of a 130 Watt ultrasonic processor (SONICS VC130PB, Sonics and Materials Inc., USA) equipped with a sonication probe 3 mm in diameter. The sonication was carried out at amplitude 80 (13 W). Before treatment sample was poured into beaker surrounded by water with ice. The durations of the ultrasonic treatments were: 0, 10, 30 minutes, 1, 1.5, 2, 4 and 6 hours. HA was optionally recovered by lyophilisation.

Gel Permeation Chromatography

Gel Permeation Chromatography (GPC) was performed using a Waters 410 refractive index detector with GPC for class VP (Version 1.02) and a Plaquagel —OH mixed 8 μm 300×7.5 mm column (Polymer Laboratories Ltd., UK). The composition of the mobile phase was similar to that recommended by column's manufacturer. It was composed of 0.2M NaCl (the replacement for commonly used NaNO₃), 0.01M NaH₂PO₄ brought to pH 7.4 by NaOH solution. The flow rate was 1 ml/min, and the column and detector temperature was 35° C.

PL Polysaccharide standards (PL Polymer Laboratories, Germany) with molecular weights of 788, 404, 212 and 112 kDa were used for calibration curve. Due to the lack of high molecular weight standards, the molecular weight values exceeding 788kDa were only extrapolated by using the equation obtained from the calibration curve.

Standards and samples were dissolved in the mobile phase (5 mg/5 ml). Each determination was carries out at least three times and the mean value was given as a result.

Preparation of Hyaluronic Acid/Chitosan Nanoparticles

The HA solution prepared at a concentration of 0.1 or 0.2% w/v in deionised water and optionally ultrasonicated was mixed with chitosan solution (CL113 0.1% or 0.2%, CL213, G113, G213 0.1% w/v in deionised water) at room temperature under magnetic stirring. Stirring was maintained for 10 minutes to allow the stabilisation of the system. The suspension of nanoparticles was instantaneously obtained upon the addition of the chitosan solution to HA solution, but in some cases aggregates were formed as well. In those cases where the aggregates were formed samples were left overnight to allow the sedimentation, and the supernatant was further analysed.

Preparation of sCT-Loaded Hyaluronic Acid/Chitosan or Hyaluronic Acid/Protamine Nanoparticles

An appropriate amount of sCT was dissolved in HA solution after sonication, and then chitosan chloride (CL113) solution (0.1% or 0.2% w/v) or protamine sulphate solution (0.1% or 0.2% w/v) was added drop wise to HA/sCT solution. sCT concentrations in the final nanoparticles suspension were: 0.1, 0.2, 0.35, 0.5 and 1 mg/ml, depending on the formulation. Stirring was maintained for 10 minutes to allow the complete formation of the nanoparticles. Nanoparticles were formed instantaneously upon the addition of the CL113 or protamine solution to HA or HA/sCT solution, but in some cases larger particles (possibly micron-sized aggregates) were formed.

sCT Loading Studies

Non-associated sCT was separated from nanoparticles by a combined ultrafiltration-centrifugation technique (Centriplus YM-50, Millipore, USA). 5 ml of sample were placed in the sample reservoir (donor phase) of the centrifugal filter device and centrifuged for 1 hour at 3000 rpm. After centrifugation the volume of the solution from the filtrate vial (acceptor phase) was measured with the use of automatic pipette and the filtrate was assayed for the content of sCT by HPLC (non-associated sCT). The nanoparticle suspension from sample reservoir was made up to 5 ml with deionised water and 1 ml was further centrifuged for 30 minutes at 13000 rpm in order to disintegrate the particles and release sCT. The sCT content in supernatant was assayed by HPLC (associated sCT). The rest of nanoparticles from sample reservoir were analysed for particle size, zeta potential and transmittance at 500 nm. The particle size, zeta potential and transmittance at 500 nm (to determine the optical density/concentration of nanoparticles) as well as the pH and viscosity of nanoparticles before the separation of non-associated sCT were also measured.

sCT Release Studies

1 ml of sCT-loaded nanoparticles collected in the filter after loading studies were suspended in 9 ml of PBS. 2.5 ml of the suspension was withdrawn for analysis at the first time point and the release medium was separated from nanoparticles using filtration/centrifugation. Retained nanoparticles were re-suspended in 2.5 ml with fresh PBS and the studies continued. At each time point, the whole 2.5 ml was separated from the release medium and then reconstituted with PBS up to 2.5 ml. The amount of sCT released at each time point was assayed by HPLC.

Viscosity

Low frequency vibration viscometer (SV-10 Vibro Viscometer, A&D Company, Limited) was employed to measure the viscosity of polymer solutions and nanoparticles suspensions. Samples were equilibrated to 25° C. in the water bath (Precision Scientific Reciprocal Shaking Bath Model 25) prior to the measurement. Three batches were done for each sample; for one batch each determination was carried out at least three times and the mean value was taken as a result.

Fourier Transform Infra-Red Spectroscopy (FTIR)

Fourier transform infer-red spectroscopy was used in order to examine the changes in the hyaluronic acid after sonication as well as the polymer interaction in polyelectrolyte complexes. Infrared spectra were recorded on a Nicolet Magna IR 560 E.S.P. spectrophotometer equipped with MCT/A detector, working under Omnic software version 4.1. A spectral range of 650-4000 cm⁻¹, resolution 2 cm⁻¹and accumulation of 64 scans were used in order to obtain good quality spectra. KBr disks method was used with 0.5-1% sample loading. KBr disks were prepared by direct compression under 8 bar pressure for 2 minutes.

Particle Size and Zeta Potential Analysis

Samples were placed directly into the folded capillary cells without making any dilutions. Each analysis was carried out at 25° C., the equilibration time was 5 minutes. The readings were carried out at least three times for each batch and the average values of at least three batches have been presented. The mean particle size and polydispersity index of the nanoparticles were determined by Dynamic Light Scattering (DLS) also known as Photon Correlation Spectroscopy (PCS), and the zeta potential values were measured with the use of Laser Doppler Velocimetry (LDV). Both DLS and LDV analysis were performed with size and zeta potential particle sizer Zetasizer Nano series Nano-ZS ZEN3600 fitted with a 633 nm ‘red’ laser (Malvern Instruments Ltd., UK). Viscosity values was taken into account when particle size and zeta potential were measured.

Morphology

Transmission electron microscopy (TEM) (Jeol 2100, Japan) was used in order to examine the morphology of nanoparticles. The samples were immobilised on copper grids and stained with either 1% w/v ammonium molybdate solution for 60 seconds or 1% w/v uranyl acetate solution for 30 seconds and dried overnight for viewing by TEM.

pH Measurements

The pH of the nanoparticles suspension was also measured, as pH is the most important factor that affects zeta potential. An Orion pH meter (model 520A) equipped with an Orion Ross™ 8103SC glass body pH semi-micro electrode was used for pH measurements. The pH meter was calibrated using standard buffer solutions (Orion) of pH 4.00, 7.00 and 10.00 (±0.01).

pH Titrations

The properties of nanoparticles (e.g. particle size and zeta potential) are highly dependent on the pH of the medium. Particle sizer Zetasizer Nano series Nano-ZS ZEN3600 fitted with a 633 nm ‘red’ laser together with MPT-2 autotitrator (Malvern Instruments Ltd., UK) were used in order to examine the influence of pH on the size and zeta potential of the nanosuspensions. 0.1M and 0.1M NaOH were used as titrants. 12 ml of sample were initially added to the sample container. Each analysis was carried out at 25° C. in automatic regime. Three particle size and three zeta potential measurements were carried out for each pH value and sample was recirculated between repeat measurements. Each analysis was started at current pH and the end pH values were 2 and 9, the increment was 0.5, and target pH tolerance was of 0.2. The isoelectric point of the nanoparticles was also determined.

1. A polymeric nanoparticle comprising at least one polycationic polymer; at least one polyanionic polymer; and a therapeutically effective amount of at least one therapeutic agent.

2. The polymeric nanoparticle of claim 1, in which the at least one polycationic polymer, and the at least one polyanionic polymer, are each a biodegradable and biocompatible polymer.

3. The polymeric nanoparticle of claim 1, in which the at least one polycationic polymer, and the at least one polyanionic polymer, are each a polysaccharide; optionally are each a mucoadhesive polysaccharide.

4. The polymeric nanoparticle of claim 1, in which the at least one polycationic polymer is a protein, optionally a protamine.

5. The polymeric nanoparticle of claim 1, in which the at least one polycationic polymer is selected from chitosan, or a salt, ester or derivative thereof; and protamine or a salt, ester or derivative thereof.

6. The polymeric nanoparticle of claim 1, in which the at least one polycationic polymer is chitosan chloride salt or chitosan glutamate salt.

7.-11. (canceled)

12. The polymeric nanoparticle of claim 1, in which the at least one polyanionic polymer is hyaluronic acid or a salt, ester or derivative thereof.

13.-16. (canceled)

17. The polymeric nanoparticle of claim 1, in which the polymeric nanoparticle comprises the at least one polyanionic polymer and the at least one polycationic polymer in a ratio of about 2.5:1-10:1 (by weight).

18. The polymeric nanoparticle of claim 1, in which the at least one therapeutic agent is water soluble and is optionally selected from low molecular weight chemotherapeutic agents; biological agents; and macromolecular chemotherapeutic agents.

19. The polymeric nanoparticle of claim 1, in which the at least one therapeutic agent is a polypeptide hormone molecule selected from the calcitonin family or a propeptide thereof.

20. (canceled)

21. A method for the preparation of a polymeric nanoparticle of claim 1; the method comprising the steps of:
i. admixing the at least one polyanionic polymer with the at least one therapeutic agent; and
ii. introducing to the mixture of (i) to the at least one polycationic polymer.

22. The method of claim 21, in which the at least one polyanionic polymer is provided in a solution of about 0.1-0.2% (w/v).

23. The method of claim 21, in which the at least one polyanionic polymer, is subjected to agitation prior to the admixing step.

24.-26. (canceled)

27. The method of claim 21, in which the at least one polycationic polymer is provided in a solution of about 0.1-0.2% (w/v).

28.-33. (canceled)

34. The method of claim 21, in which the polymeric nanoparticle is prepared under conditions, which are substantially free of surfactant.

35. A method for treating an inflammatory and/or arthritic disorder caused by or associated with dysfunctional nuclear receptor signaling, the method comprising administering a polymeric nanoparticle according to claim 1.

36. The method of claim 35, in which the nuclear receptor is an orphan nuclear receptor.

37. The method of claim 35, in which the nuclear receptor is selected from NR4A1 (nuclear receptor subfamily 4, group A, member 1); NR4A2 (nuclear receptor subfamily 4, group A, member 2); and NR4A3 (nuclear receptor subfamily 4, group A, member 3).

38.-42. (canceled)

43. A method for treating a disorder caused by or associated with dysfunctional matrix metalloproteinase signaling, the method comprising administering a polymeric nanoparticle according to claim 1.

44. The method of claim 43, in which the matrix metalloproteinase has collagenase activity.

45.-47. (canceled)
